(19) United States
(12) Patent Application Publication
(10) Pub. No.: US 2025/0003014 A1
(43) Pub. Date: Jan. 2, 2025

(54) MicroRNA Sensor

(71) Applicant: Board of Supervisors of Louisiana State University and Agricultural and Mechanical College, Baton Rouge, LA (US)

(72) Inventors: Caroline Copeland, Redwood City, CA (US); Yongchan Kwon, Baton Rouge, LA (US)

(73) Assignee: Board of Supervisors of Louisiana State University and Agricultural and Mechanical College, Baton Rouge, LA (US)

(21) Appl. No.: 18/689,519

(22) PCT Filed: Nov. 13, 2023
(86) PCT No.: PCT/US2023/079456

§ 371 Date: Mar. 6, 2024

Related U.S. Application Data
(60) Provisional application No. 63/425,509, filed on Nov. 15, 2022.

(51) Int. Cl. C12Q 1/6897; C12Q 1/6876

(52) U.S. Cl. CPC C12Q 1/6897; C12Q 1/6876; C12Q 2600/178

(57) ABSTRACT

A microRNA sensing platform is disclosed that is sensitive, accurate, easy to use, affordable, and that does not require expensive equipment on the part of the end-user. The sensing system can be used as a sensitive and accurate miRNA profiling platform to monitor the progression of diseases, or treatment regimen, or personalized diagnosis, or personalized prognosis. The entire RNA-induced silencing complex (RISC), which holds the mature miRNA, is preserved in the system. The RISC is allowed to perform its native functions. These native functions not only protect the miRNA from degradation during the isolation process, but can also help eliminate false targets. The system sensitively and selectively responds to the presence or absence of RISCs bearing the target miRNA.

The benefit of the 15 Nov. 2022 filing date of U.S. provisional patent application Ser. No. 63/425,509 is claimed under 35 U.S.C. § 119(e) in the United States, and is claimed under applicable treaties and conventions in all countries.

This invention was made with support from the United States Government under grant 2020-94414-21535 awarded by the National Institute of Food and Agriculture. The government has certain rights in the invention.

### TECHNICAL FIELD

This invention pertains to improved methods for detecting and quantitating microRNA.

### BACKGROUND ART

There is an unfilled need for improved methods for early diagnosis and monitoring of diseases and infections. Studies have reported that the dysregulation of microRNA (miRNA) is correlated with the development of and with the specific stages of various diseases, including multiple cancer types, viral infections, diabetes, and cardiovascular diseases. The miRNAs, discovered in 1993, are a type of small, non-coding RNA that accounts for ˜1-3% of the mammalian genome. Over 1900 miRNAs have been reported to play various functions, some of them critical. The miRNAs have been reported to play a role in regulating the expression of at least half of the human transcriptome.

Many approaches have been used to detect circulating miRNA, including Northern blot, in situ hybridization, RT-PCR, microarray, and Next-Generation Sequencing. However, these methods of detecting miRNA have not been standardized, and their results are not always consistent. Also, prior miRNA detection methods have generally required lengthy and expensive procedures that generally must be performed in a well-equipped laboratory. There is an unfilled need for an miRNA sensing platform that is reliable, quick, accurate, inexpensive, and that can be deployed by end users without the need for a well-equipped laboratory.

What is miRNA?

microRNAs (miRNAs) are small, non-coding RNA molecules first discovered in Caenorhabditis elegans in 1993. miRNA can be transcribed from DNA sequences with their own promoters (intergenic), or they can be processed from introns (intragenic) into primary miRNA (pri-miRNA). Several nearby miRNA loci can constitute a polycistronic transcription unit. Alternatively, multiple miRNAs can be co-transcribed from clusters to create a long transcript. A cluster is a family of miRNAs with similar seed regions. The seed region is the domain of the miRNA near the 5′-end comprising primarily the nucleotides at the second through the seventh position. The seed region is the main binding region for miRNA in eukaryotic cells. An miRNA can bind to the 3′-untranslated region (UTR) of the mRNA, the 5′-UTR, the coding sequence, the promoter, or other regulatory regions.

There are at least two miRNA biogenesis pathways. In the canonical pathway, a pri-miRNA (a ˜1 kb stem-loop structure) is processed into pre-miRNA by a combination of the RNA binding protein called DiGeorge Syndrome Critical Region 8 (DGCR8), and the Drosha ribonuclease III enzyme. DGCR8 locates the N6-methyladenylated GGAC motif, among others, while Drosha cleaves the base of the hairpin. The newly formed pre-miRNA is then exported to the cytoplasm by exportin 5, bound to the protein Ran-GTP. The pre-miRNA is released by hydrolysis of the GTP bond. After export, the pre-miRNA is cleaved near the terminal loop by the Dicer RNase III endonuclease, releasing a mature miRNA duplex. The mature miRNA duplex is then loaded onto an Argonaute protein (AGO), with help from chaperone machinery, to form an RNA-Induced Silencing Complex (RISC).

What is called the “guide strand” of an miRNA is determined by the thermodynamic stability of its 5′ and 3′ strands. The strand with lower stability, typically the 5′ strand with a 5′ uracil at position 1, is deemed the guide strand. It is the guide strand that is loaded onto an Argonaute protein molecule (AGO). The name of the active miRNA includes a designation for which strand was loaded as the guide strand, viz. −5p or −3p.

The miRNA molecules bind to target mRNA sequences by complete or incomplete base pairing. Binding at the 3′ UTR normally represses translation, and induces deadenylation and decapping. Binding at the 5′ UTR or to a coding region silences the targeted genes. Interactions with promoters can induce (or can inhibit) transcription. Translation is activated when AGO2 and miRNA associate with the protein FXR1 and travel to AU-rich elements (AREs) at the 3′ UTR. GW182 proteins work in tandem with AGO to silence mRNA. Once an miRNA has bound to an AGO protein, sometimes an interaction with proteins GW182 and PABPC occurs that triggers deadenylation, which in turn represses the mRNA or leaves it prone to degradation by exonucleases. There are also additional pathways through which miRNA-RISC complexes regulate gene expression.

The miRNA molecules can be released into various extracellular fluids, to become so-called circulating miRNA. miRNAs tend to be rather stable as compared to other RNA molecules. They can resist degradation at room temperature for up to four days, and they can survive relatively harsh conditions. Circulating miRNA can be found in vesicles (exosomes, apoptotic bodies, etc.) and protein complexes (e.g., AGO). miRNA could potentially be used as a biomarker for various diseases. There is an unfilled need for improved methods to detect and quantitate miRNA.

Circulating miRNA

Circulating miRNA generally remains associated with AGO proteins. This association enhances miRNA stability.

Recent studies have suggested that cell-to-cell communication can be mediated by the transport of miRNA in extracellular vesicles (EVs). This cell-to-cell communication is readily observed in certain cancers. For example, there have been observations that when exosomes from triple-negative breast cancer cells (MDA-MB-231s) were incubated with non-triple negative cells (estrogen receptor-positive MCF-7s), the two miRNAs were overexpressed in the MDA-MB-231 exosomes, downregulating tumor suppressor genes in MCF-7s, and demonstrating that extracellular exosomes can alter gene expression. Research has also shown that exosomes can sometimes contribute to a recurrence of cancer. For example, after the primary tumor has been cleared from a breast cancer patient, metastatic tumors sometimes develop from circulating cells that have lain dormant, sometimes even years later. Such dormancy can be induced by exosomes from bone marrow mesenchymal stem cells (MSCs) communicating with the metastatic breast cancer cells via exosomes that carry the miRNA miR-23b.

There have been several reports of miRNA transfer via exosomes in breast cancers and other cancers, and other associations of miRNA with various disease states, angiogenesis, and transfer of drug resistance. Communication via miRNA in exosomes has also been observed in the immune system.

miRNA molecules can not only travel cell-to-cell within an organism, but they also can travel between organisms, and even between organisms of widely separated taxa, typically as part of an attack on a host or by a host. For example, the stomach parasite Heligmosomoides polygyrus suppresses host immunity by transporting its miRNA in exosomes to host epithelial cells to suppress genes involved in inflammation and immunity. These types of transfer can even occur in viral infections, with infected cells sending miRNA in vesicles to non-infected cells, to induce changes in their immune responses.

### MicroRNA Target Binding Mechanism: RISC, AGO, and Beyond

The RISC comprises a mature miRNA that serves as the guide, and an AGO protein that provides the action. When bound to a target, miRNA can only form 1-2 helical turns, which does not release much binding energy from base pairing alone. Binding to the target and helical turning of the miRNA are aided by the AGO protein. The AGO protein has a lysine residue that neutralizes the negative charge of the bound miRNA at positions 1 and 3, assisting the guide to bind to the target without negative charge repulsion between their respective backbones. AGO also displays the seed nucleotides of the miRNA in a pre-helical conformation, which lowers the thermodynamic barrier for target binding.

The manner in which the miRNA is held in AGO, and the manner it binds to its RNA target promotes high specificity. If an initial seed match to a potential RNA target is not fully complimentary, the RISC complex releases the target and moves on to search for a new target in approximately one second. This mechanism allows for high specificity and low error rates. After the seed sequence has bound, high complementarity between the miRNA and the target results in stronger binding and an extended time for the duration of the bound complex. High complementarity usually results in a cleavage event of the target mRNA via AGO2 protein. The RISC is presumably adapted not to damage a transcribed RNA unless there is a high degree of certainty that it has indeed identified its proper target.

The most abundant miRNA molecules appear to regulate translation by target repression rather than by endonucleolytic cleavage, which explains why miRNAs are expressed in relatively large amounts—namely, because it takes the RISCs longer to repress RNA than it would take to cleave it.

Prior Methods for Assaying microRNAs

The most common ways to detect and measure miRNA have included Northern blot, in situ hybridization, RT-PCR including qRT-PCR, microarray, and Next-Generation Sequencing. However, these methods have not always given consistent results, and for the most part the methods have not been standardized. There is an unfilled need for improved miRNA detection methods, having improved sensitivity and specificity, methods that do not require the use of lengthy procedures, expensive processes, or require the end user to have access to a well-equipped laboratory.

qRT-PCR methods can be sensitive, but their specificity depends strongly on the particular primers used. Microarrays can suffer specificity problems because they deploy a large set of probes simultaneously, some of which may have similar sequences and experience crosstalk. There is also high variability across microarray platforms from different manufacturers.

Other approaches that have been used or suggested for miRNA sensing include isothermal amplification, electrochemical methods, enzymatic reactions, various biochemical reactions, and nanoparticles. Unfortunately, many of these methods are susceptible to false-positives and have inadequate specificity. High specificity is important in making a proper diagnosis or prognosis, particularly in view of the high homology seen across various miRNAs sequences. However, many prior methods for miRNA detection have relied solely on the base-pairing of short, single-stranded, mature miRNAs. Such hybridization methods can lead to problems where, for example, a probe binds to a similar but non-identical miRNA, making it hard to assess the accuracy of a putative signal.

Conventional Technologies for miRNA detection (summary)

| Method | Description | Cost | Pros | Cons |
|---|---|---|---|---|
| Northern | miRNA in sample is size-separated by | $$$$ | Can detect | High cost, labor- |
| blotting | gel electrophoresis, and then cross- | | both mature | intensive, time |
| | linked to a membrane. Complementary | | miRNA and | consuming, |
| | synthetic nucleic acid probes bind | | precursors. | potentially |
| | miRNA on the membrane. | | Detects both | hazardous |
| | | | sequence and | (sometimes uses |
| | | | length | radioactive ³²p) |
| RT-qPCR | Gold standard for quantifying circulating | $$$ | Most well- | Time-consuming, |
| | miRNA. There are some commercial | | equipped labs | complicated, |
| | assay products including: miRCURY | | already have | requires |
| | LNA qPCR from Exiqon, TaqMan | | the necessary | expensive |
| | assays from Applied Biosystems/Fisher | | equipment, | equipment, and a |
| | Thermo Scientific, and two-tailed RT- | | aside from the | well-equipped |
| | qPCR miRNA assays from TATAA | | probes. The | laboratory. |
| | Biocenter. | | primers and | The possibility for |
| | Since miRNAs are short RNA | | probes offer | contamination |
| | molecules, additional sequence is | | much | and error exists |
| | typically added at the 3′ end to all | | versatility. | within both |
| | miRNAs in a sample. Then a universal | | | amplification |
| | primer binds this tail, and reverse | | | steps. |
| | transcription proceeds. Other options | | | Sensing probes |
| | include stem-loop RT primers, linear RT | | | are expensive, |
| | primers, DNA pincer probe, and two- | | | suffer from high |
| | tailed RT primer. However, these | | | and variable |
| | primers must be specifically tailored for | | | background |
| | the miRNA of interest. | | | noise, and must |
| | | | | go through a |
| | | | | complicated |
| | | | | design process to |
| | | | | be made efficient. |
| | | | | Difficulty in |
| | | | | repeating |
| | | | | quantitative |
| | | | | results due to the |
| | | | | exponential |
| | | | | nature of PCR |
| | | | | amplification. |
| fluorescent | Fluorescent-labeled nucleic acid probes | $$$ | Preserves | Low throughput, |
| in situ | that can detect single stranded RNA or | | RNA integrity, | but can detect |
| hybridization | DNA in tissue or fixed cells. However, | | many options | multiple miRNAs |
| (FISH) | the binding of the probes is poor. | | for signal | simultaneously. |
| | Locked nucleic acid modifications are | | enhancement, | Locked nucleic |
| | therefore often used, which can be | | can monitor | acid probes help |
| | costly. | | cellular and | binding, but |
| | | | subcellular | cause high non- |
| | | | distribution | specific binding |
| | | | | and can be |
| | | | | expensive. |
| microarrays | Small nucleic acid probes | $$$ | Can quantify | Variability across |
| | complementary to the miRNA targets | | many | platforms and |
| | are immobilized on glass slides via | | microRNAs | between labs is |
| | crosslinking. The isolated potential | | simultaneously | high. |
| | miRNA targets are labeled with | | | Off target binding |
| | fluorescent dye, so that when | | | can occur. |
| | hybridization occurs with a probe, a | | | Can be |
| | fluorescence signal is emitted. The | | | expensive, |
| | reaction can be sensitive to annealing | | | especially when |
| | temperature. Unfortunately, miRNAs are | | | trying to inhibit |
| | short and don't readily allow for | | | off-target binding. |
| | temperature optimization, which can | | | Requires a well- |
| | result in off-target annealing or no | | | equipped |
| | annealing at all. Commercial platforms | | | laboratory. |
| | have been made by Agilent | | | |
| | Technologies, Ambion Inc., Exiqon, | | | |
| | Invitrogen, Thermo Fisher Scientific, | | | |
| | and others. Some popular products | | | |
| | include miRCURY LNA microRNA | | | |
| | array, Luminex FlexmiR microRNA | | | |
| | Human Panel array, and GeneChip | | | |
| | miRNA Array. | | | |
| Next | Can determine the specific sequence of | $$$$$ | Can detect | Very expensive. |
| generation | target microRNA by ligating 3′ and 5′ | | unknown | Requires heavy |
| sequencing | linkers, reverse transcription, and PCR | | microRNAs. | computation for |
| | amplification. The nucleotides are linked | | Has higher | data analysis and |
| | to 4 different fluorescent dyes so the | | accuracy and | interpretation. |
| | sequence can be determined as | | sensitivity than | |
| | nucleotides are added. | | other | |
| | | | conventional | |
| | | | methods. | |

Non-Conventional Technologies for miRNA detection (summary)

| Method | Description | Cost | Pros | Cons |
|---|---|---|---|---|
| Isothermal | This technique amplifies nucleic acid | $$ | Less expensive | Because these methods are |
| amplification | sequences at a constant | | and simpler to | isothermal, they can suffer |
| | temperature, instead of cycling the | | conduct the | from low accuracy. Binding |
| | temperature as in PCR. Methods | | reactions, as | of the miRNA is the main |
| | include rolling circle amplification | | compared to qRT- | trigger, but that binding is |
| | (RCA), exponential amplification | | PCR. | highly dependent on using |
| | reaction (EXPAR), hybridization | | | the proper annealing |
| | chain reaction (HCR), catalytic | | | temperature. Because the |
| | hairpin assembly (CHA), strand- | | | reaction is exponential, any |
| | displacement amplification (SDA), | | | off-target binding can give a |
| | duplex-specific nuclease signal | | | large false response. These |
| | amplification (DSNSA), and loop- | | | methods also suffer from |
| | mediated isothermal amplification | | | self-activation, i.e., |
| | (LAMP). In most of these methods, | | | producing a significant |
| | the miRNA acts as a primer. As the | | | background signal even |
| | template is amplified, a fluorescent | | | when no sample is present. |
| | dye intercalates between the two | | | Also, the enzymes, |
| | strands, which can then be read by a | | | specialized oligonucleotide |
| | fluorescence reader. | | | probes, and dyes tend to |
| | | | | make the methods complex |
| | | | | and expensive to implement. |
| Nanoparticle- | Nanoparticles, most commonly gold | $$ | High surface area | Difficult to achieve both high |
| based | nanoparticles, are used in | | to volume ratio, | sensitivity and high |
| detection | conjunction with a detection element, | | multiplexing | specificity due to crosstalk, |
| | and a signal output method. | | capabilities, | autofluorescence, and |
| | Nanoparticles have been used to | | possible point-of- | reaction inhibition. Also, |
| | quench fluorescent dyes attached to | | care use | most nanoparticle detection |
| | DNA probes. The DNA probe is | | | devices use DNA probes to |
| | attached to a nanoparticle. miRNA | | | capture the miRNA, and off- |
| | binding to the DNA probe, or opening | | | target binding can be an |
| | a DNA hairpin, or triggering an | | | issue. The costs of |
| | enzymatic cleavage reaction, can | | | manufacturing the |
| | separate the FITC dye from the | | | nanoparticles and |
| | nanoparticle, producing a | | | specialized DNA probes or |
| | fluorescence signal. Some | | | antibodies can be high. |
| | nanoparticle clusters, such as silver | | | |
| | nanoclusters (AgNCs), can | | | |
| | themselves fluoresce. Fluorescent | | | |
| | quantum dots (QD) have been used | | | |
| | to detect miRNA by fluorescence | | | |
| | resonance energy transfer (FRET) | | | |
| | between the QD and a DNA probe. | | | |
| | In another approach, two originally- | | | |
| | separated components of a | | | |
| | fluorophore are brought together by | | | |
| | streptavidin (e.g., attached to the | | | |
| | QD) and biotin (e.g., attached to the | | | |
| | capture probe), which is released | | | |
| | once the miRNA binds. | | | |
| Nano- | Solid surfaces generate a response | $$ | Ability to | Surface reactions can |
| structured | when miRNA from a sample binds to | | concentrate the | trigger background |
| surfaces | immobilized DNA probes on the | | analyte on the | reactions, since the reaction |
| | surface. Reactions can use surface | | surface to reach | is not localized. Concerns |
| | plasmon resonance. | | higher | exist about the specificity of |
| | | | sensitivities. | DNA probe hybridization. |
| | | | | The materials to build and |
| | | | | conduct these reactions can |
| | | | | be costly and prone to user- |
| | | | | error. |
| Electro- | Electrochemical-based biosensors | $$ | Readily | Specialized oligonucleotides |
| chemical- | can convert mechanical energy to | | miniaturized; can | for each reaction can be |
| based | electrical energy in various ways: | | be used in point of | expensive to manufacture, |
| systems | amperometric, potentiometric, | | care applications. | as can antibodies. Also, |
| | impedimetric, or ion charge/field | | Manufacture is | many reactions rely on |
| | effect. Most rely on DNA/RNA | | inexpensive. | nucleic acid hybridization, |
| | hybridization, which is operatively | | Enhanced | which can be sensitive to |
| | connected to an electrical read-out | | scalability of | the annealing temperature. |
| | system. The probe can be | | readout devices | |
| | immobilized either on nanoparticles | | as compared to | |
| | or on a flat conductive surface. One | | optical-based | |
| | type of reaction that can be triggered | | devices. | |
| | for detection is redox reaction. Other | | | |
| | systems include a magnetosensor, | | | |
| | and magnetic beads attached to | | | |
| | DNA/RNA probes to hybridize to | | | |
| | target miRNA. | | | |
| Microfluidics | The flow of liquids is controlled in | $$ | Increases output | Melting temperatures of |
| | small channels, over millisecond time | | signal by enabling | miRNA vary from 45-72° C., |
| | periods, to process reactions at | | the user to | but only one temperature |
| | different times in separate | | separate the | can be used during each |
| | compartments. Miniaturized and | | reactions, | reaction, which leads to a |
| | automatic FISH reactions are | | allowing greater | potential source of error. |
| | possible. | | manipulation and | |
| | | | therefore | |
| | | | permitting the | |
| | | | design of a more | |
| | | | complex reaction. | |
| | | | Can readily be | |
| | | | miniaturized. Can | |
| | | | be inexpensive, | |
| | | | because the | |
| | | | components can | |
| | | | be made of simple | |
| | | | plastics or even | |
| | | | from paper. | |
| Lateral flow | LFA-based assays are centered | $ | Easy to use and | Specialized oligonucleotides |
| assay | around chromatographic separation, | | manufacture, | can be expensive to |
| (LFA)- | primarily capillary forces in paper- | | potential point of | manufacture for each |
| based | based devices, so that the sample | | care use. | reaction, as can antibodies. |
| | with the target miRNA flows past a | | | Many of these reactions rely |
| | reaction hub and triggers a response. | | | on nucleic acid |
| | These reactions are based on | | | hybridization, which can be |
| | nucleic acid hybridization with an | | | very sensitive to the |
| | immobilized probe or an immobilized | | | annealing temperature. |
| | antibody. The probe includes some | | | Low signal intensity, mainly |
| | modification, such as biotin or HRP, | | | useful for qualitative |
| | that triggers a cascade of events to | | | applications. |
| | produce a signal. | | | |

C. Copeland, The Design of a microRNA Sensor using an Improved Cell-Free Protein Synthesis System for Low-Limit Biomarker Detection (Louisiana State University Doctoral Dissertation Abstract 6127, Apr. 5, 2023) is an abstract of a Dissertation by one of the inventors, summarizing some of the same work reported here. The Dissertation was defended on Mar. 21, 2023. The Abstract has been publicly accessible online since Apr. 5, 2023. However, the full Dissertation itself is not yet publicly accessible, and will only become publicly accessible on Apr. 3, 2030.

Heitzer, E. et al. The potential of liquid biopsies for the early detection of cancer. npj Precision Oncology 1, 36 (2017) is a review article discussing the use of circulating tumor DNA in early cancer diagnosis. The potential of using circulating miRNA for a similar purpose is also mentioned.

The use of circulating microRNA as a diagnostic for cancer and other diseases, or even for therapeutics is discussed in Chen, X. et al. Characterization of microRNAs in serum: a novel class of biomarkers for diagnosis of cancer and other diseases. Cell Research 18, 997-1006 (2008); Anfossi, S. et al. Clinical utility of circulating non-coding RNAs—an update. Nature Reviews Clinical Oncology 15, 541-563 (2018); Rupaimoole, R. et al. MicroRNA therapeutics: towards a new era for the management of cancer and other diseases. Nature Reviews Drug Discovery 16, 203-222 (2017); and Mitchell, P. S., et al., Circulating microRNAs as stable blood-based markers for cancer detection. Proceedings of the National Academy of Sciences, 2008. 105(30): p. 10513-10518.

Detassis, S. et al. microRNAs Make the Call in Cancer Personalized Medicine. Frontiers in Cell and Developmental Biology 5 (2017) is a review article discussing the use of microRNAs in cancer diagnosis and therapy. The authors noted that a limitation in existing microRNA detection methods was the closely similar sequences among microRNA family members. The main detection methods used, qRT-PCR, ddPCR, microarrays, and NGS were said to lack strong sensibility and accuracy, especially at single-base resolution. The authors recognized an unfilled need for a “more accurate and PCR-free single base sensitive platform.”

Pritchard, C. C. et al. MicroRNA profiling: approaches and considerations. Nature Reviews Genetics 13, 358-369 (2012)) is a review article discussing the use of microRNA profiling for various purposes, including developmental biology, diseases biomarkers, and forensics. The authors mentioned that sample processing and RNA extraction methods can have a substantial impact on the results of miRNA profiling, particularly for samples that are prone to miRNA degradation. The advantages and disadvantages of qRT-PCR, microarrays, and other methods are outlined.

Ouyang, T. et al. MicroRNA Detection Specificity: Recent Advances and Future Perspective. Analytical Chemistry 91, 3179-3186 (2019) is a review article discussing methods used for detecting miRNA molecules, such as Northern blot, reverse transcription quantitative polymerase chain reaction, next-generation sequencing, and microarray. The authors noted an unfilled need for improved specificity in detection, and methods that were being attempted to enhance specificity. See also Kilic, T., et al., microRNA biosensors: Opportunities and challenges among conventional and commercially available techniques. Biosensors and Bioelectronics, 2018. 99: p. 525-546.

Reviews of cell-free expression systems, their uses in biosensing applications including RNA sensing, and other uses to which cell-free systems have previously been put, can be found in Silverman, A. D. et al. Cell-free gene expression: an expanded repertoire of applications. Nature Reviews Genetics 21, 151-170 (2020); and in Copeland, C. E. et al. The cell-free system: A new apparatus for affordable, sensitive, and portable healthcare. Biochemical Engineering Journal 175, 108124 (2021)

The RISC complex, its assembly, and its functions are described in Yoda, M. et al. ATP-dependent human RISC assembly pathways. Nature Structural & Molecular Biology 17, 17-23 (2010). The authors also reported that human RISC assembly is uncoupled from dicing, and is fueled by ATP.

Klein, M., et al., Why Argonaute is needed to make microRNA target search fast and reliable. Semin Cell Dev Biol, 2017. 65: p. 20-28 gives an experimental and theoretical discussion of the search process used by RISCs and Argonaute proteins, along with miRNA, to specifically identify cognate messenger RNA for degradation. See also Wee, L. M., et al., Argonaute divides its RNA guide into domains with distinct functions and RNA-binding properties. Cell, 2012. 151(5): p. 1055-1067.

### DISCLOSURE OF THE INVENTION

We have discovered a microRNA sensing platform that can detect any selected miRNA or miRNAs. For example, the novel platform may be used to screen a full miRNA profile to identify patterns associations with various disease onsets and progressions. The novel platform is sensitive, accurate, easy to use, affordable, and does not require expensive equipment on the part of the end-user. The costs of manufacturing and storage become very inexpensive due to the small volumes of reagents needed, inexpensive reagents, and low-maintenance conditions for storing the reagents and carrying out the reactions.

The entire RNA-induced silencing complex (RISC), which holds the mature miRNA, is preserved. The RISC is allowed to perform its native functions. These native functions not only protect the miRNA from degradation during the isolation process, but they can also help eliminate false positive results. The platform uses an ultrasensitive “circuit” or cascade in a cell-free expression system. Notwithstanding that the miRNA-AGO combination works in vivo as a complex, prior miRNA sensing methods have not used the entire RISC, but instead have focused on isolating and detecting the miRNA alone. But isolating just the miRNA can have the unintended consequence that informative data is discarded. When the miRNA molecule is isolated, it is no longer associated with a complex whose function is to aid in the complete and accurate binding of miRNA to its target, and to protect miRNA from degradation enzymes such as RNases. Despite these disadvantages, to our knowledge no previous detection method has employed the entire RISC for detection, but they have instead employed isolated RNA molecules. Prior methods have focused on using the mature miRNA only, and have discarded the rest of the complex that encases the miRNA, the full RISC. It can be more difficult to perform downstream assays with miRNA when it is collected in the RISC form, which may have led prior researchers away from developing assays based on the full complex.

One embodiment is based on an Escherichia coli cell-free gene expression (CFE) system. Other cell-free expression systems known in the art may also be used. The system provides a versatile in vitro biosensing platform. When freeze-dried, the entire system can be kept in low-maintenance storage until use.

Circulating RISCs may be initially isolated by methods known in the art, for example, with a centrifuge and filter. A cell-free expression system “circuit” to detect miRNA preferably comprises a rapidly-responding, low-leak repression system, one that by default goes immediately into the OFF-state when the target RISC-miRNA is absent, and that sensitively and promptly switches to the ON-state when and only when the target RISC-miRNA is present.

The switching action can, for example, be implemented with a sigma/anti-sigma repression system such as the ECF11_987 sigma factor, along with its cognate anti-sigma factor and its cognate promoter region, all from the bacterium Vibrio parahaemolyticus. These factors have no significant crosstalk with endogenous E. coli factors and promoters. A preferred reporter protein is one of the brightest fluorescent proteins currently used in the art, mNeonGreen (mNG).

When the system is in the ON state, mNG gene transcription initiates once a circulating RISC-miRNA binds to a complementary region upstream of the anti-sigma coding sequence on a plasmid, thereby disrupting transcription of anti-sigma, either by blocking ribosomes or by slicing the sequence, which allows more sigmas to be unsequestered. The E. coli RNA Polymerase (endoRNAP) core enzyme then binds the cognate DNA promoter of the ECF11_987 sigma factor, which controls expression of the mNG fluorescence reporter gene.

See FIG. 1, which schematically depicts one embodiment of a simple circuit based on the ECF11_987 sigma factor. The sigma11 and anti-sigma11 DNA coding sequences sig11 and anti-sig11 are under the control of the upstream promoter PT7. PT7 is a very strong promoter. The two plasmid vectors containing the sig11 and anti-sig11 coding sequences are prepared in large batches, purified, and supplied to the cell-free reaction. Also included in the cell-free reaction is the cell extract. The cell extract is made, for example, from the E. coli BL21 Star DE3, which overexpresses the T7 RNA polymerase (T7RNAP) during culture. The culture is harvested, lysed, and clarified to form the cell extract. At the start of the cell-free reaction, the numerous bacteriophage T7RNAPs already present in the system promote the expression of both anti-sigma11's and sigma11's from mRNA transcripts in the extract.

In the OFF State, when no pertinent miRNA/RISC complex is present, anti-sigma is expressed. The sigma coding sequence is under the control of another copy of the PT7 promoter, and sigma is also expressed. However, the anti-sigma binds the expressed sigma. The bound sigma is then unavailable to activate transcription of the mNG. The net effect is that the mNG reporter molecule is not expressed. When no pertinent miRNA/RISC is present to bind to the miRNA complementary region (CR) between the anti-sigma's ribosome binding site (RBS) and the start codon, the anti-sigma gene translation is not disrupted, allowing anti-sigma molecules to sequester sigma11 molecules, so that sigma11 cannot bind to its cognate promoter, which controls mNG expression; and the E. coli endogenous RNA polymerase cannot bind sigma11 to start transcription of mNG.

In the ON State, the pertinent miRNA/RISC is now present. The complex binds the miRNA complementary region (CR), which is upstream of the anti-sigma coding sequence. The bound RISC disrupts transcription of anti-sigma. Without anti-sigma to block it (or at reduced levels of anti-sigma), the sigma11 binds its cognate promoter PS11, and the E. coli endogenous RNA polymerase (endoRNAP) binds to the sigma and transcribes the mNG coding sequence, which is then expressed to produce the mNeonGreen (mNG) reporter molecule by ribosomal machinery. The net effect is that when the pertinent miRNA/RISC complex is present, the fluorescent reporter molecule is expressed, and can then be assayed either qualitatively or quantitatively. The disruption of transcription by the RISC-miRNA is strongly sequence-dependent, allowing high confidence that an observed ON signal means that the miRNA indeed contains the RNA sequence of interest.

FIG. 2 depicts an optional but preferred embodiment that also employs an amplifier. Signal detection is amplified, for example by adding a DNA construct containing the ECF11_987 sigma factor promoter to control transcription of the pseudo-sigma portion of a T7-CCG-split pseudo-sigma factor. When transcribed, the pseudo-sigma factor couples with the larger of the split pieces, the “T7 core,” which has previously been expressed, purified, and added to the reaction mixture.

The “split” T7 RNAP is known in the art. The native T7RNAP is split into two pieces: a larger “core” piece, and a smaller “pseudo sigma” piece. The smaller pseudo-sigma sequence has previously been operatively linked to a promoter sequence different from that used by the unmodified whole T7RNAP. The two components of the T7 RNAP must both be present to activate transcription at the T7 cognate promoter. Splitting the expression of the T7 polymerase thus has the effect of a transcriptional “AND” gate.

The “amplifier circuit” depicted in FIG. 2 acts similarly to that of FIG. 1, with modifications: Once the ratio of sigma to anti-sigma is disrupted, free sigma11s bind to their cognate promoter, which in turns activates transcription for the coding sequence of the T7-split CGG pseudo-sigma protein, and thus activating the circuit's amplifying module. This binding then allows the T7-split CGG sigma to bind its cognate promoter (PCGG) and transcribe the coding sequence for the reporter molecule mNG. mNG is then expressed to produce a large visual or measurable response.

The circuit thus can allow for amplification after the target miRNA has been detected, which reduces the incidence of false negatives and increases sensitivity. Adding the amplifier after the sensing module, rather than before, helps reduce false positives. The novel circuit also has a very low “leakage” rate, i.e., sigma11 proteins that are produced even without the presence of the correct miRNA-RISC. The leakage rate is sufficiently low that it does not prematurely trigger the amplification cascade (at least not to an extent comparable to that when a correct miRNA is present), so that background noise is thus very low. Because the expression system is cell-free, it can tolerate substances in concentrations that might be toxic to or otherwise complicate detection in systems based on living cells. The circuit can readily use blood samples and other samples with minimal purification, which lowers costs. Also, the sensor is able to reach low and competitive detection limits. In tests to date, we have detected samples at concentrations as low as 81 pM, with a limit of quantification as low as 271 pM.

The system (or its components) can optionally be freeze-dried, making the system inexpensive to transport and to store with a long shelf life. The freeze-dried platform is readily rehydrated. The inexpensive components and user-friendly operation of this system are conducive to frequent testing, which can help improve monitoring of a patient's disease state, testing access for low-income families, and testing access in rural areas.

Advantages, features, and optional features of the novel system include the following:
1) Using the entire RISC-miRNA complex in detecting circulating miRNA: Using the entire RNA-induced silencing complex (RISC) not only protects the miRNA from degradation during isolation, but also helps reduce off-target detection (false positives) by taking advantage of the native functions of the RISC. So far as we are aware, the system reported here is the first to base miRNA detection upon the entire RISC, rather than on isolated RNA. Maintaining the intact RISC enhances specificity. The Argonaute proteins and the RNA work together to enhance the specificity of RNA-RNA binding. The novel technique can successfully distinguish between a complete match and a single base mismatch, something that even PCR cannot do consistently. The Argonaute proteins also help protect against the various mechanisms that tend to degrade RNA.
2) Using a cell-free system to detect the entire RISC-miRNA complex for circulating miRNA detection: Employing the full functions of the RISC works best in a cell-like working environment. A cell-free protein synthesis (CFPS) system, or a cell-free gene expression (CFE) system offers an ideal environment for using the benefits of the RISC for circulating miRNA sensing.
3) Using a novel repression circuit to detect a RISC-miRNA in a CFE system, for example based on a non-native sigma/anti-sigma pair: A preferred embodiment employs the repression of a non-native sigma by a non-native anti-sigma factor. Then, when a targeted RISC-miRNA analyte is present, repression is broken, and the non-native sigma allows transcription of a reporter gene.
4) Use of a split T7 system for high signal sensing: A preferred embodiment employing an amplifier based on the split T7 system is believed to be the first report of using a split version of a robust bacteriophage RNA polymerase to improve gain in a biosensor circuit. Using a split RNA polymerase offers control and modularity. The split polymerase only functions when the “missing” component is produced by the circuit, triggered by the specific analyte.

Using a cell-free system allows the RISCs to operate much as they do inside a cell. Once the cell-free medium has been prepared initially, there is no further requirement to culture any cells as part of the assay. The novel approach should be less expensive to implement than currently-used detection methods, such as qRT-PCR. Also, PCR is subject to errors when there are small mismatches in a sequence. The novel system is superior to PCR at detecting small mismatches, even those as small as a single base.

Anti-sigma represses sigma. When not repressed by anti-sigma, sigma promotes the expression of a reporter molecule, such as a fluorescent protein. The sigma factor is preferably from a different bacterial system (e.g., Vibrio versus E. coli), to minimize crosstalk with components of the cell-free system.

In one embodiment, the cell-free system includes a robust T7 RNA polymerase. This polymerase transcribes both sigma RNA and anti-sigma RNA in vitro. The T7 polymerase does not need a sigma factor to initiate transcription. By contrast, the endogenous E. coli RNA polymerase needs a sigma factor in order to bind initially to the promoter to begin transcription. T7 is a stronger polymerase than endogenous polymerases.

The optional amplifier allows more sensitive detection of target miRNA molecules at clinically-relevant levels. An optional amplifier circuit uses, for example, a “split” T7 polymerase to add a selective robust transcription of reporter protein after initial accurate sensing. The “split” condition makes the OFF state more robust, by allowing a small amount of initial sigma to be used. The “split” also makes the ON state more robust, by amplifying the signal exponentially when the target analyte is present. The “split” polymerase remains inactive until its two components bind to one another.

The system can be tuned in various ways to reduce noise, for example by adjusting the ratio of sigma to anti-sigma. Tuning the ratio of sigma and anti-sigma factors helps conserve energy in the cell-free system, as compared to making more polymerase, thus making the system more efficient.

### Cell Free Protein Synthesis (CFPS)

The novel method employs a cell-free protein synthesis system. The RISC complex requires either a cellular environment or an environment mimicking a cellular environment to function efficiently. To enhance efficiency and to reduce the cost of the sensor, an E. coli cell-free protein synthesis system (CFPS), also known as a cell-free system (CFS), was chosen as the basic preferred sensing platform. E. coli are fast-growing and can produce high levels of protein. Cell-free systems based on E. coli are easy to tune with plasmids and supplements, making design-build-test cycles fast and low-cost.

Our preferred CFPS system comprises an in vitro transcription-translation system derived primarily from cell-free extract from lysed E. coli cells. The cells' endogenous DNA and mRNA are removed, and the crude extract is supplemented with appropriate components, e.g., suitable amino acids, nucleotides, cofactors, salts, DNA, or mRNA. Proteins can be synthesized in a CFPS with less concern over the effects of cell walls, over whether bacterial growing conditions are favorable, or over toxic byproducts. A cell-free system allows for rapid production of protein, until one of the substrates is depleted or until a byproduct reaches an inhibitory concentration. An in vitro cell-free system can accomplish in ˜1-2 days what might require ˜1-2 weeks in an in vivo system. E. coli-based systems can yield from a few micrograms up to several milligrams of protein per batch, depending on the particular protocol used.

Prior cell-free genetic circuit cascades have generally started the system in the ON state. However, to enhance sensitivity with low analyte concentrations, we found it preferable to start in a strong OFF state. Also, we have employed a very bright fluorescence reporter molecule, to enhance detection of weak signals.

### Orthogonal Sigma and Anti-Sigma Factors to Enhance Sensitivity

In one aspect, the invention enhances regulation to support a reliable, sensitive, inexpensive, and robust miRNA sensing platform. A strong OFF state is achieved through the use of sigma and anti-sigma protein-protein interactions, which thus regulates promoters. Sigma factors are prokaryotic proteins having a dissociable subunit of RNA polymerase that binds to a specific promoter region of DNA, which in turn allows the RNA polymerase to bind to create a holoenzyme and to transcribe the DNA. Anti-sigma factors are a related transcriptional control mechanism. Anti-sigmas are proteins that bind to sigma factors, inhibiting the sigmas from binding to DNA, and thus inhibiting binding of the RNA polymerase to the promoter, with the consequence that transcription is inhibited.

In one embodiment a strong OFF transcriptional state results from factors including these: 1) strong protein-protein interactions of the sigma/anti-sigma combination, resulting in a strong OFF state for the entire circuit; 2) expression of the reporter protein begins in the OFF state, because sigma is needed to begin transcription; 3) very low leakage, because the sigma/anti-sigma system presumably evolved to prevent malformation and death of the cell, unlike repressor transcription factor operons which presumably evolved for cellular energy conservation; 4) the ability to tune the concentrations of sigmas and anti-sigmas, for example by controlling plasmid concentration.

The system provides ultrasensitivity, and readily generates a strong sigmoidal response. Switching from the OFF state to the ON state is rapid. Because repression is based on protein-protein interactions, rather than on protein-DNA interactions as in most prior approaches, any delay in switching from the OFF state to the ON state essentially vanishes. The switch from OFF to ON can occur almost instantaneously when the ratio of the pertinent miRNAs shifts. Although sigma/anti-sigma regulation has been tested in whole-cell studies to demonstrate its strong OFF state capability, to our knowledge sigma/anti-sigma regulation has not previously been used in a CFE system, nor has it previously been used in microRNA sensing applications.

The sigma factor used in a prototype embodiment was an alternative extracytoplasmic function (ECF) sigma factor, one that is non-native to E. coli. ECF sigma factors respond to signals that might be generated outside the cell or in the cell membrane. Most ECF sigma factors are controlled by corresponding anti-sigma factors. ECFs are a large and diverse group. ECFs exhibit high target promoter specificity, and low non-specific DNA binding activity. The ECF sigmas will function appropriately in diverse organisms, because RNAP subunits are strongly conserved across bacterial taxa. The specific ECF sigma chosen for certain embodiments of the invention was an ECF11 sigma factor from the bacterium Vibrio parahaemolyticus (also known as ECF11_987). The ECF11_987 sigma and its cognate anti-sigma AS11_987 exhibit high repression and low cross-reactivity. We selected the orthogonal pair of sigma ECF11_987 and anti-sigma ECF11_987 (AS11_987) for one embodiment, along with P11_₃₇₂₆, an ECF11-specific promoter. To minimize potential leakage, it is preferred that the anti-sigma and sigma coding sequences should be transcribed and translated independently. Independent expression allows the control of one without affecting the other, for example by silencing anti-sigma RNA translation without disturbing sigma RNA translation.

### The Lyophilized (Freeze-Dried) Platform

Many reactions relying on living cells require refrigeration during much or all of the distribution process, known as cold-chain distribution, which can increase cost. Also, solution-phase reactions are sometimes not well-suited for use outside a laboratory setting, due to potential environmental contamination and other practical difficulties. Cold-chain and solution-phase distribution problems can be avoided with cell-free systems, reducing the high cost and complications that come with delicate storage needs. Lyophilization of a cell-free reaction system allows on-demand, point-of-care use in diverse settings. With proper freeze-drying, function can be maintained at a high level when the system is reconstituted. This capability allows the system to be used outside a conventional laboratory, in a biosafe manner, helping to extend access to healthcare to low-income households, remote communities, third-world countries, combat zones, and victims of natural disasters.

### Discussion

The novel miRNA sensing platform is the first of its kind in several ways. Using a cell-free expression (CFE) system, the entire RNA-induced silencing complex (RISC) with a mature miRNA retains the ability to perform its native functions, resulting in large enhancements in specificity. The RISC can induce an ultrasensitive circuit cascade. Prior miRNA detection platforms have overlooked the advantages that can be derived from retaining the entire RNA-induced silencing complex. Using the entire RISC not only helps protect the miRNA from degradation during isolation, but it also reduces off-target detection by taking advantage of the native functions of the RISC. To employ the full functions of the RISC it is preferred to use a cell-free system. Prototype demonstrations have successfully identified targets with a limit of detection at 81 pM, and a limit of quantification at 271 pM.

In one embodiment the system initially employs sigma repression by a non-native anti-sigma factor. When the target RISC-miRNA analyte is present, sigma repression is broken, and the non-native sigma is released to transcribe a reporter gene. In an optional embodiment, the system also synthesizes one piece of a split, robust, phage-derived RNA polymerase to amplify the signal. The RNA polymerase can then transcribe a sequence corresponding to a readily-detectable reporter molecule, such as a bright fluorescent protein.

We believe this to be the first report of using this type of gene disruption as a sensor in a CFE system, as well as the first time a split RNA polymerase has been used in this manner. Leakage from the sensor is sufficiently low that it may simply be subtracted from the total signal at the differentiating level, even after the reaction has run to completion. “Leakage” here means any mNG that is synthesized without the presence of the proper miRNA, resulting for example from the dissociation of the sigma from the anti-sigma, or from a lag in association following translation. Such leakage may be subtracted because it typically will not reach the same endpoint fluorescence value as a true signal from a correct miRNA sequence. A cell-free system can be tolerant to potentially toxic substances. The circuit can use samples such as blood samples with minimal purification, lowering costs.

Tests may be run inexpensively. When the platform is freeze-dried, it is shelf-stable. The novel detection system out-performs many prior miRNA detection methods, such as RT-qPCR, Next Gen Sequencing, microarrays, and non-conventional assays. The novel system features accuracy, consistency, portability, simplicity, and low cost.

In an alternative embodiment, purified miRNA is loaded into a pre-purified Argonaute protein, and the resulting complex can then be used in the invention as otherwise described herein. The principal difference lies in initially purifying the miRNA without its endogenous Argonaute protein. This separation can perhaps lead to more degradation and handling errors, but the overall process may also be more convenient; it is easier and less expensive to purify RNA alone than an RNA-protein complex. The pre-purified Argonaute proteins for use in this alternative embodiment can be made recombinantly, and can be derived from any of several species. However, the miRNA loading preferences of the particular Argonaute molecule should be kept in mind, as some perform better with double-stranded miRNA, while others prefer single-stranded miRNA. Keeping the loading preference for single- or double-stranded RNA in mind, miRNA from one species can be loaded into an Argonaute from a different species for this alternative embodiment.

### BRIEF DESCRIPTION OF THE DRAWINGS

FIG. 1 depicts schematically one embodiment of the simple circuit using the ECF11_987 sigma factor.

FIG. 2 depicts schematically an embodiment of the circuit employing an amplifier.

FIG. 3 depicts respective sigma factors' activity with the endogenous RNA polymerase in the CFPS reaction, as compared to that of T7 RNA polymerase.

FIGS. 4(a) and (b) depict the results of measurements to seek optimal plasmid concentrations in a system with multiple RNA polymerases.

FIG. 5 depicts fluorescence levels at different ratios of anti-sigma DNA to sigma DNA in the cell-free system.

FIG. 6 depicts the effects of adding, into a CFPS circuit reaction mixture, miRNA loaded into purified Argonaute (AGO) protein (RISC-miRNA).

FIG. 7 depicts three time-course assays, as well as RISC circuit specificity, for three samples: a target miRNA (miR-155), an miRNA with a single base mismatch (155-mm8), and an unrelated miRNA (cel-39).

FIGS. 8(a), (b), and (c) depict the outcome of assays carried out with the novel system and with RT-qPCR for comparison.

FIG. 9 depicts three amplifier candidates' activity in the CFPS reaction, compared to that with the ECF11 sigma factor but no amplifier.

FIG. 10 depicts the results of certain CFPS reactions testing the T7-CGG split core and sigma factor.

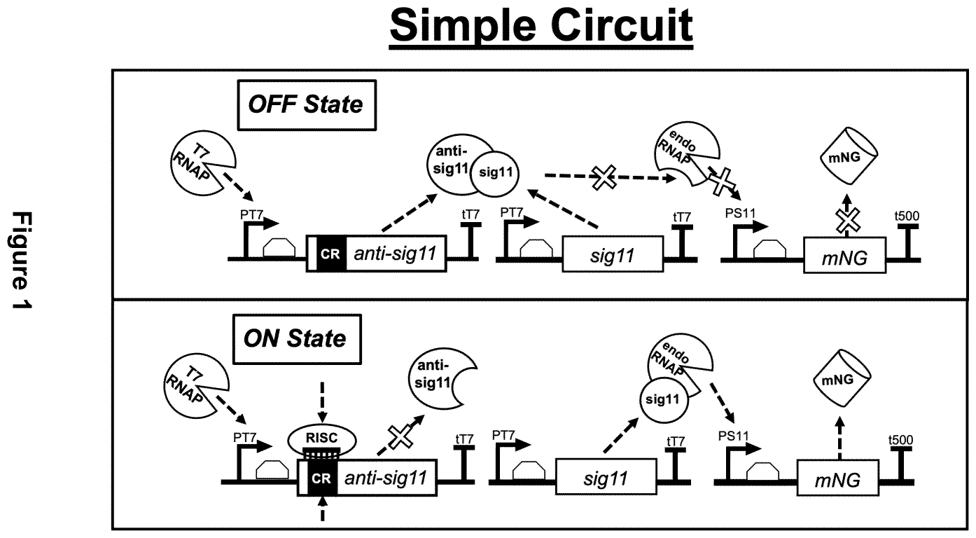

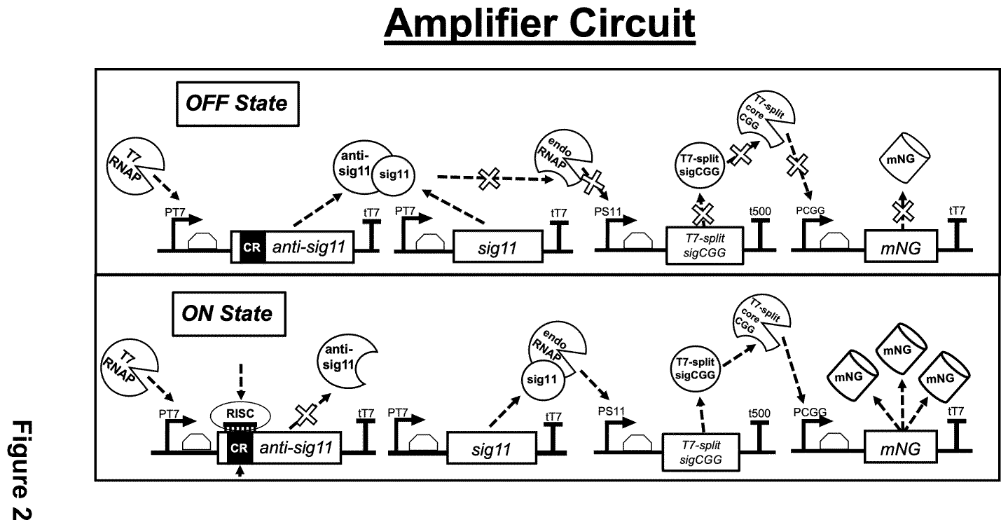

### MODES FOR CARRYING OUT THE INVENTION

### Example 1. Robust Sigma Factor

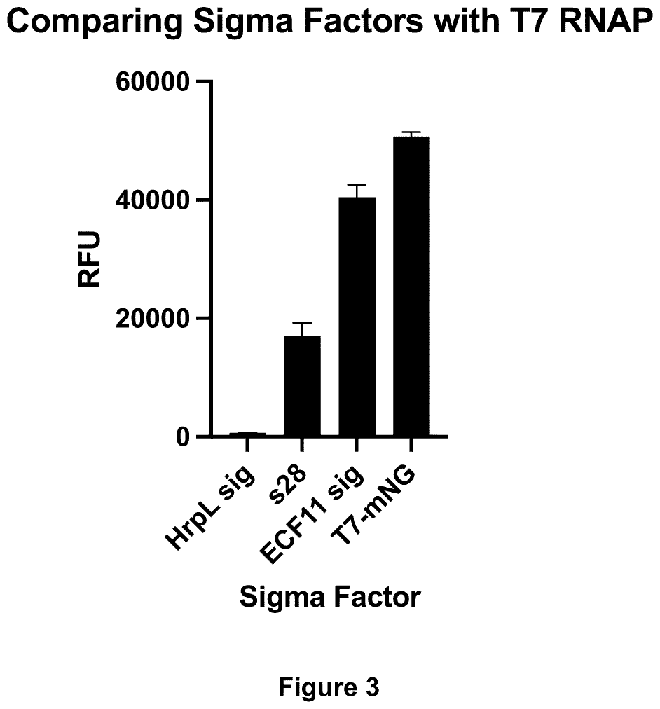

A sigma factor is a protein needed to initiate transcription in bacteria. The sigma factor enables specific binding of RNA polymerase to a promoter. The specific sigma factor employed depends on the particular gene. We screened three different sigma factors, and compared their activity to that of T7 RNA polymerase. All sigma factors were expressed with T7 RNA polymerase in the cell-free reaction system. We tested a well-known, highly-active, endogenous E. coli sigma factor, sigma 28; a new sigma factor not known to have been previously tested in genetic circuitry, HrpL from Pseudomonas syringae; and another sigma factor that had previously been shown to have high activity in whole-cell systems, sigma factor ECF11_987 from Vibrio parahaemolyticus. A cognate promoter for each sigma factor was placed upstream of a sequence encoding mNeonGreen fluorescent protein. Relative to the T7 RNAP expression of mNG, ECF11_987 produced a large and significant difference in expression as compared to the other sigma factors, demonstrating its robust activity. FIG. 3 depicts the respective sigma factors' activity in the CFPS reaction as compared to that of T7 RNA polymerase. Each reaction included a plasmid in which the sigma factor coding sequence was controlled by T7RNAP, and also a plasmid containing the mNG gene controlled by the particular sigma factor's cognate promoter. Values are represented as mean±SD, n=3.

Because the prototype cell-free reaction used a mixture of endoRNAP and T7 RNAP, and because those two polymerases are of differing strengths, we sought to optimize the DNA concentration for each plasmid (pJL1-T7-sig11 and pJL1-Ps11-mNG) to enhance overall performance. We found it preferred for the concentration of T7-sig11 plasmid DNA to be low as compared to the concentration of pJL1-Ps11-mNG plasmid DNA.

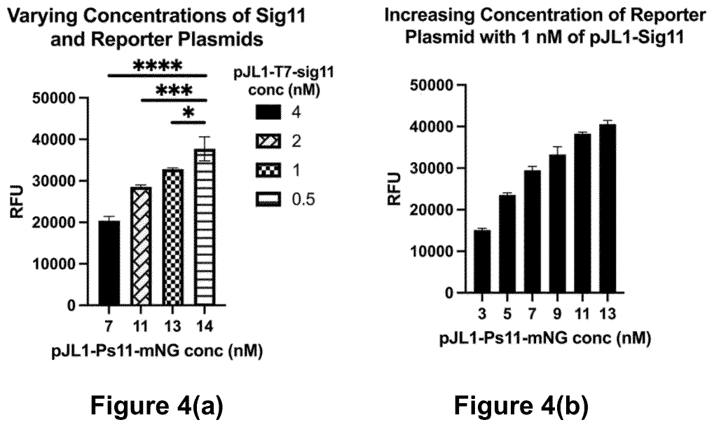

FIGS. 4(a) and (b) depict the results of measurements to seek optimal plasmid concentrations in the system with multiple RNA polymerases. FIG. 4(a) shows the effect of varying the concentrations of the plasmid expressing sigma factor ECF11 (pJL1-T7-sig11) and of the reporter plasmid expressing mNG under the cognate sigma11 promoter (pJL1-Ps11-mNG). Values are represented as mean±SD, n=3. ****p<0.0001, ***p<0.001, *p<0.05. FIG. 4(b) shows the effect of the concentration of the reporter-bearing plasmid pJL1-Ps11-mNG, and its evident saturation. Values are represented as mean±SD, n=3.

The output of mNG from pJL1-Ps11-mNG began to saturate at a DNA concentration around 13 nM. The sigma ECF11 has a cognate anti-sigma that inhibits binding of the sigma to its cognate promoter. We used this interaction in the circuit's repression module. To ensure that the repression interaction was robust, we expressed both genes on separate plasmids, each controlled by T7 RNA polymerase; and also placed the sigma11 cognate promoter upstream of an mNG coding sequence on a different plasmid. The repression from anti-sigma was extremely strong, reaching saturation at a molar ratio of anti-sigma DNA to sigma DNA around 2:1 to 4:1.

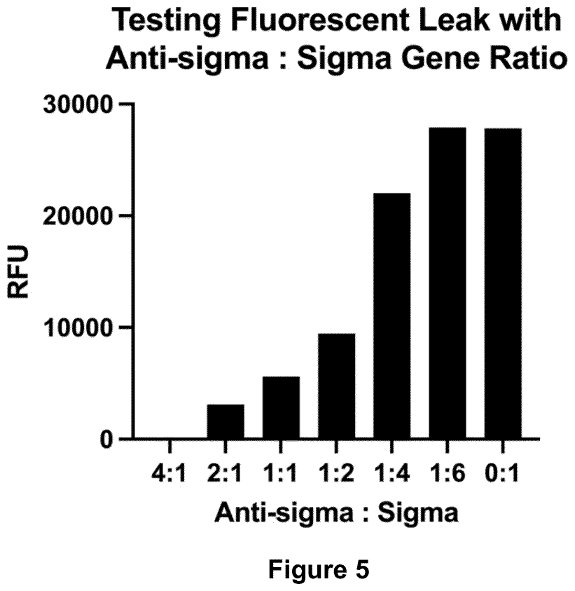

FIG. 5 depicts fluorescence levels, and thus repression activity of the ECF11 anti-sigma factor, at different ratios of anti-sigma DNA to sigma DNA in the cell-free system. Plasmids with the anti-sigma factor coding sequence and plasmids with the sigma factor coding sequence, both under the control of T7RNAP, were added to the reaction. A plasmid containing the mNG coding factor, under control of the ECF11 sigma factor's cognate promoter, was also present in each reaction. Activity for the different combinations is shown as RFU (relative fluorescence units). Values are represented as mean±SD, n=3. The data in FIG. 5 represent endpoint measurements after 20 hours. Repression was essentially 100% at an anti-sigma to sigma ratio of 4:1, without significant leakage after 20 hours reaction time.

### Example 2. The miRNA-RISC Detection Circuit

Using the sigma/anti-sigma pair along with sigma 11's cognate promoter, we constructed an miRNA-RISC detection gene circuit. The anti-sigma and sigma coding sequences were each monocistronic, to facilitate fine control of the ratio of the expressed sigma and anti-sigma factors. In particular, the two coding sequences were placed on two separate plasmids. To inhibit repression within the circuit, the miRNA-RISC complex silences the anti-sigma coding sequence by targeting an miRNA complementary region (CR) just downstream of the start codon for the anti-sigma sequence. RISC bound to the anti-sigma mRNA induces ribosome dissociation from the anti-sigma mRNA. The RISC can also expose endocatalytic activity at the CR. The anti-sigma-to-sigma ratio then becomes unbalanced, allowing free sigma molecules to bind to the mNG promoter, thereby initiating transcription of mNG and generating a fluorescence signal. By translating sigma and anti-sigma independently, and using separate ribosome binding sites for the two, anti-sigma RNA translation can be silenced without disturbing that for sigma.

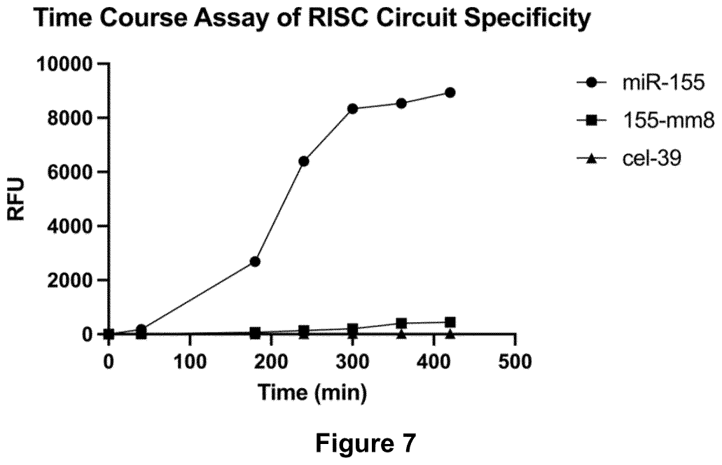

In the OFF state (i.e., in the absence of miRNA-RISC), the circuit remains strongly repressed, and mNG is not expressed. When the pertinent miRNA-RISC is present, the circuit expresses mNG continuously; only a few sigmas need to be expressed to permit synthesis of the mNG mRNA; the result is that the signal becomes brighter over time, as illustrated in FIG. 7.

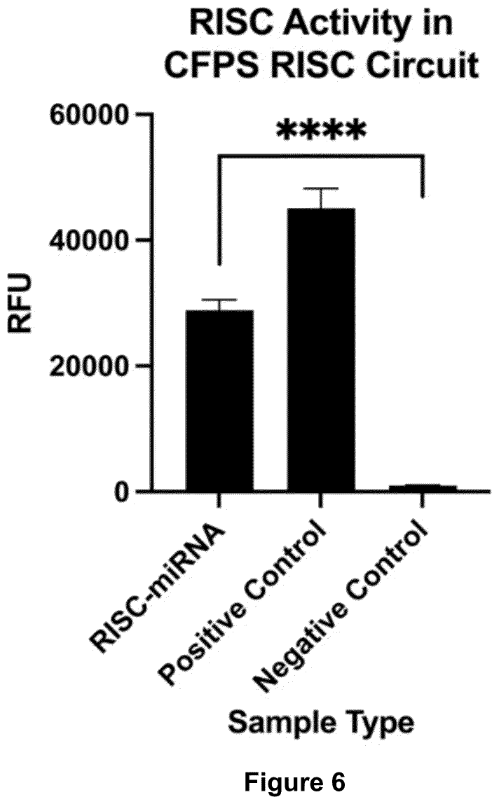

FIG. 6 depicts the effects of adding, into a CFPS circuit reaction mixture containing all the pertinent Simple Circuit sequences, miRNA loaded into purified Argonaute (AGO) protein (RISC-miRNA). The positive control contained the RISC-miRNA with the ECF11 sigma factor coding sequence only, but without the anti-sigma ECF11 repressor. The negative control contained the Simple Circuit components and AGO protein, but without miRNA. The final concentration of miRNA in the CFPS circuit was 13.33 ng/μL, and that of AGO protein was 19.33 ng/μL. The reaction mixture had a volume of 15 μL. Values are given as mean±SD, n=3, ****p<0.0001.

SDS-PAGE analysis confirmed that the resulting protein expression was consistent with our expectations, with the circuit proteins and the mNG proteins expressed where expected, and not otherwise. (data not shown)

We carried out time-course assays to observe when the mNG could be first detected and when its expression halted or slowed, depending on the particular miRNA loaded into the AGO protein. FIG. 7 depicts three such time-course assays, as well as RISC circuit specificity, for three samples: a target miRNA (miR-155), an miRNA with a single base mismatch (155-mm8), and an unrelated miRNA (cel-39). Relative fluorescence units (RFU) were measured at different time points. FIG. 7 shows that expression became noticeable around 100 minutes, and approached its maximum around 300 minutes. FIG. 7 also shows that off-target triggering occurred later, and at a much lower RFU with a single nucleotide mismatch; and not at all for an unrelated miRNA. For the target miRNA, a strong green fluorescence signal was easily visible to the naked eye at 300 minutes, while nothing was visible to the naked eye for the single-base mismatch, nor for the unrelated RNA. These data illustrate an advantage of the present system, its ability to distinguish target RNA from an otherwise-identical RNA having as little as a single base mismatch. Even PCR has difficulty accurately distinguishing a target from a non-target molecule with a single base mismatch.

In one embodiment, the cell-free free reaction components are freeze-dried along with the circuit plasmids. The system is later reconstituted for use with an miRNA-RISC sample. As a quality check, fluorescence over time is measured as compared to a non-freeze-dried reaction, to determine how long the system reagents can be satisfactorily stored in a freeze-dried state.

### Example 3. Loading Purified hAGO2 with Synthetic Single-Stranded miRNA

To verify that the circuit functioned as intended, and to further test its performance, we constructed an artificial RISC by loading single-stranded miRNA into purified human Argonaute-2 (hAGO2). Recombinant human Argonaute-2 protein (AGO2 or hAGO2), the “slicer” in RNA interference, was expressed in Sf9 insect cells using a baculovirus expression system, following otherwise standard procedures. The expressed AGO2 was immunopurified, using a FLAG-tag fused to the protein. The AGO2 was then observed as a highly pure band in a Coomassie Blue-stained SDS-PAGE gel near a 100 kDa marker (data not shown).

The single-stranded mature miRNA was purchased from Integrated DNA Technologies (IDT), synthesized with a 5′-end phosphate modification to facilitate proper RISC loading. Following previous loading protocols, we assembled the RISC complex and observed its activity. The miRNA loading saturation point, assayed as maximum RFU in the cell-free simple circuit reaction in the presence of 200 nM AGO2 protein, was observed to be around 1750 nM (data not shown).

To better optimize the RISC's activity in the CFS, we varied the amount of cell extract, magnesium glutamate, pH, and temperature. Our observations gave the following preferred conditions: −12 mM final concentration magnesium glutamate, ˜7.8 pH HEPES buffer, ˜33.5° C. reaction temperature, and 26.7% concentration of cell extract for the cell-free circuit containing the amplifier module.

Our prototype embodiments have used human Argonaute protein 2, but other types of Argonaute protein known in the art will also work in practicing the invention. Several types of human Argonaute protein that carry microRNA have been reported in the literature, and additional types from other taxa are known as well, including those from various mammals, plants, and insects. Some Argonaute proteins cleave the target sequence, while others simply bind to it, but in either case they disrupt RNA translation. Those variations that do not cleave RNA may still be used in the novel circuit: They bind to the target sequence in the mRNA, and a ribosome translating that mRNA will fall off the mRNA, so that translation will cease.

### Example 4. Characterizing the Efficiency of the Novel Circuit, Compared to RT-qPCR

RT-qPCR (reverse transcription-quantitative polymerase chain reaction) has generally been considered the “gold-standard” for detecting miRNA. The nucleic acid hybridization methods used in RT-qPCR allow relatively low incremental costs for carrying out the reactions (after the relatively high fixed cost of the initial equipment purchases have been incurred). RT-qPCR is amenable to standardization of the procedures used from purification to detection. Some commercial miRNA diagnostics have used RT-qPCR. Due to the inherently exponential nature of PCR amplification, there can be substantial variability between test runs.

For a biomarker detection method to be clinically useful, its limit of detection (LOD) and dynamic range should be clinically relevant. Many potential biomarkers are present only in low concentrations in typical biopsy samples such as blood or serum. We tested various concentrations of an miRNA-RISC complex both with the novel platform described here, and with RT-qPCR for comparison. The samples tested also included mismatch samples, to assay selectivity.

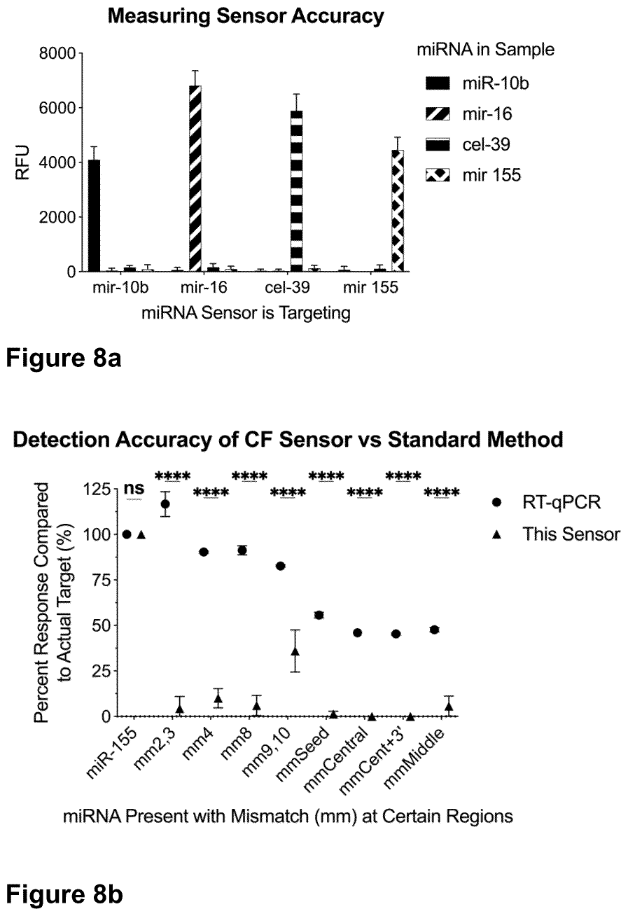

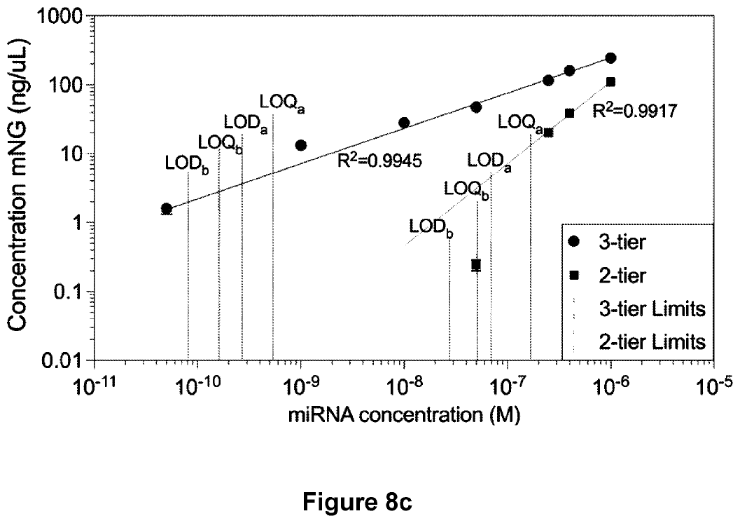

FIGS. 8(a), (b), and (c) depict the outcome of assays carried out with both the novel system and with RT-qPCR for comparison. Different concentrations and different types of miRNA were tested.

FIG. 8(a) depicts fluorescence measurements with the novel cell-free system using different RNA in the RISC, and different targets. Observed cross-reactions were very low, almost to the point of being non-existent.

FIG. 8(b) depicts a comparison of the accuracy of the sensor to that of RT-qPCR in differentiating between a target miRNA and other RNA molecules with similar sequences. The nucleotides from the correct miRNA (miR-155) were altered at various positions, measured from the 5′ end. Also, entire miRNA regions were changed, such as the seed region, central, larger middle, and central+3; end. These changes would cause mismatches with the complementary region target, and are labeled with mismatch and position number or region. Accuracy was calculated as: [1.00−(mNG output with the particular miRNA (x-axis)]/[mNG output with the correct miRNA (miR-155))]×100%. Values are given as mean±SD, n=3. ****p<0.0001.

FIG. 8(c) depicts the observed limit of detection (LOD) and limit of quantification (LOQ) for both the 3-tier circuit and the 2-tier circuit (i.e., with amplification and without, respectively) using miR-155 at varying concentrations. The LOD and the LOQ were both determined by comparison to measurements with a blank sample (designated “b” in this figure) and with a sample miRNA having a single-base mismatch (designated “a”). Values are represented as mean, min, and max.

In these experimental comparisons, RT-qPCR had a lower LOD than that of the novel method without the amplifier. However, the dynamic range of RT-qPCR was also low. For example, PCR was unable to differentiate the mismatch samples from the true target at low concentrations. See FIG. 8(b). The novel system displayed remarkable accuracy and was able to differentiate between different miRNA, even with just a single mismatch. As compared to the gold standard RT-qPCR, the novel system displayed much higher specificity in distinguishing different miRNAs having similar sequences.

Adding the “amplifier” to make a 3-tier circuit substantially improved the limit of detection as compared to the 2-tier reaction. The limit of detection for the 3-tier circuit with amplifier, as compared to the blank sample, was ˜81 pM. The limit of quantification was ˜271 pM. The LOD was determined as a signal:noise ratio of 3, and the LOQ was determined as a signal:noise ratio of 10. The observed LOD and LOQ figures were comparable to those that have been reported for other non-conventional miRNA detection methods. At these measured detection limits, and even without further optimization, the novel miRNA sensor can detect a target miRNA at a level as low as 0.015% of the total miRNA present in a 10 mL serum sample, at a total concentration of miRNA of 7 ng/mL; and it could quantify the target miRNA in such a sample at a level as low as 0.05% of the total miRNA.

### Example 5. Amplifying the Signal

To lower the limit of detection of miRNA-RISC complexes, an amplifier module is optionally added to the circuit, using a second transcriptional activator that is switched on by the first sigma. The amplifier should preferably exhibit no crosstalk with the endogenous components of the cell-free system, nor with other components of the circuit. We screened three potential candidates.

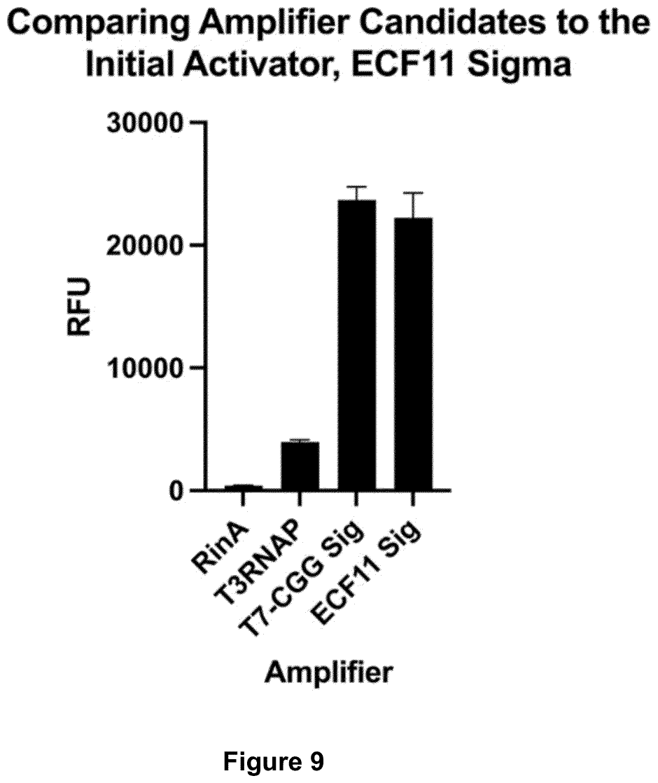

FIG. 9 depicts the amplifier candidates' activity in the CFPS reaction compared to activity with the ECF11 sigma factor but no amplifier. Each candidate reaction contained a plasmid with the amplifier sequence controlled by T7RNAP; and a plasmid containing the mNG coding sequence, controlled by the designated amplifier's cognate promoter. Values are represented as mean±SD, n=3.

The first candidate, RinA_p80α, is an ultrasensitive phage activator from Staphylococcus aureus phage 80α. It had previously been used in an amplifying module for a gene circuit in a whole cell reaction. Unfortunately, we were unable to replicate the strong response of RinA as previously reported, perhaps due to differences between whole-cell systems and cell-free systems, or perhaps due to the need for another, unidentified factor to activate transcription—some factor that naturally occurs in whole-cell E. coli but that is lost during extract preparations.

Next, we tested a robust bacteriophage RNA polymerase, T3 RNA Polymerase (T3RNAP), which had previously been used in cell-free reactions as a component in circuit cascades. Unfortunately, T3RNAP did not show as strong a response as desired, perhaps because it is such a large protein and its synthesis consumes a disproportionate share of the cell-free system resources, even when it is present in a fairly low concentration.

Third, we tested the Split-T7 RNA polymerase as described in Segall-Shapiro T H, Meyer A J, Ellington A D, Sontag E D, Voigt C A. A ‘resource allocator’ for transcription based on a highly fragmented T7 RNA polymerase. Mol Syst Biol. 2014 Jul. 30; 10(7):742. doi: 10.15252/msb.20145299. In this system the polymerase is split into two sections: a 285 amino acid DNA-binding loop, known as the ‘a fragment’; and the remaining 601 amino acid ‘core fragment’ with an attached SynZIP for better joining of the two pieces. The a fragments can be given different promoter specificities, which is useful to help minimize cross talk with the regular T7 RNAP. We chose the most robust reported a fragment having the lowest crosstalk and the highest performance, the ‘CGG sigma’ fragment, which has a CGG nucleotide binding region within the regular T7 promoter. To use the split-T7, we overexpressed the core fragment in BL21 DE3 Star E. coli in cell culture, and then continued with regular cell-extract preparations. The core fragment-rich extract was added to the cell-free reaction mixture. The CGG sigma fragment was expressed by T7RNAP. The two pieces of the T7 RNAP could join once both were present in the reaction mixture. We observed high activity after the two components had joined, comparable to that of the robust sigma11.

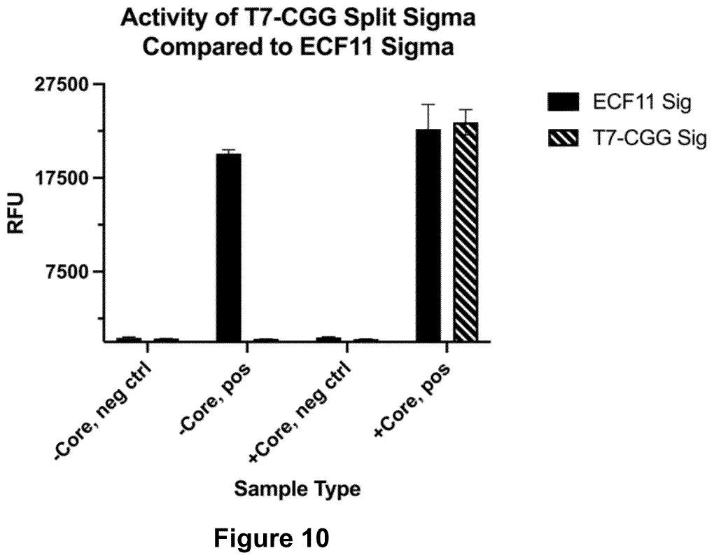

To further test the activity, we also carried out reactions with and without the core-rich extract, and with or without the CGG sigma plasmid, using a reporter plasmid (the CGG cognate promoter linked to the mNG coding sequence) as a negative control, and using sigma11 under the same conditions as positive control. FIG. 10 depicts the observed results of these CFPS reactions, testing the T7-CGG split core and sigma factor. In FIG. 10, −Core or +Core denotes whether the overexpressed T7-split core piece was present or absent. Neg ctrl or pos denotes whether the ECF11 sigma or T7-split CGG sigma was present in the CFPS reaction. The mNG reporter sequence was controlled by its cognate promoter. Values are represented as mean±SD, n=3. We observed that neither T7 RNA polymerase nor the Split T7 core fragment in the cell extract exhibited significant crosstalk with the CCG promoter (−core/+core, neg ctrl), and that CGG sigma could not activate the CGG promoter without the core (−core, pos). A strong signal was seen with T7-CGG only with +core/pos.

We then selected the Split T7RNAP system as a preferred amplifying module, and added it to the simple circuit by attaching an ECF11 promoter (PS11) to the T7-split CGG sigma gene, and then attaching the CGG sigma promoter (PCGG) to the mNG gene. See FIG. 2. After the plasmids had been assembled and purified, the LOD's were determined and compared to those for the simple circuit. The dynamic range was also determined for miRNA with a single mismatch.

### Example 6. T7 RNAP SynZip Characterization in the Cell Free System; and Quantifying Certain Preferred Reaction Conditions

The T7-split SynZip system is a preferred choice for the amplifier module. However, to our knowledge this system had never previously been used in a cell-free system, and we further characterized its properties in a cell-free system to help optimize performance of the 3-tier circuit. A preferred embodiment of the CFS miRNA sensing circuit comprises: (1) cell extract tuned for endogenous RNA polymerase, (2) plasmid encoding sigma11, (3) plasmid encoding anti-sigma11, (4) plasmid encoding the reporter protein mNG under the CGG promoter, and (5) and the T7-split core protein—which can be present either in a crude cell extract form or in a purified protein form. Other factors that can be adjusted or optimized include temperature, buffer pH (e.g. HEPES buffer), magnesium concentration, and NTP (nucleotide triphosphate) concentration.

Before further T7-split studies were carried out, plasmid expression of the core protein was re-evaluated for the specific purposes of this project. The plasmid pTHSSd_38 (#59961) was obtained from the Addgene plasmid repository. The plasmid had originally been tailored for a different purpose, namely whole cell expression, with an optimized (per cell) copy number and a goal of moderate protein expression levels. Our considerations differed from those for which the plasmid was originally designed. For example, the SynZip split reaction is not typically used in a whole cell system, the copy number is not a relevant factor in a cell-free system, and high levels of expression were sought. The pTHSSd_38 (#59961) plasmid has a low copy number in vivo. It was originally used to express genes responsible for mitigated plasmid partitioning into daughter cells. When we attempted expression in the E. coli BL21 Star™ (DE3) protein expression cell line, the Split-T7RNAP SynZip system did not appear to be active. We then tried placing the T7 core sequence into the pETBlue1 plasmid vector for overexpression, driven by the full T7 RNA polymerase. The protein was then visualized, and the SynZip T7 split system successfully expressed mNG. (data not shown)

An option was whether to use crude extract containing the T7-split core protein, or instead to use purified T7-split core protein. A benefit of using the crude extract is that it requires less labor and materials; however, in that case various post-lysis conditions should then be optimized. In one embodiment, we overexpressed the T7-split core in whole BL21 Star (DE3) cells grown to OD 3.0, and then processed the cells in the same fashion as when making a conventional cell-free extract. The lysate was tested under different post-lysis conditions, including: run-off only (+R, −D); run-off and dialysis (+R, +D), and neither (−R, −D). Observations showed that run-off only, and run-off and dialysis both hindered the SynZip interaction. Thus a preferred embodiment employed no post-lysis conditions (data not shown).

Large decreases in fluorescence also occurred in the +R, +D processed T7 core enriched extract when it was added to the 2-tiered circuit, showing that these processing steps hinder transcription or translation generally, through an unknown mechanism. (data not shown) To reduce inhibition from the core extract, the T7 core piece was purified by metal affinity purification of the N-terminus histidine-tagged T7 core protein, and was then washed several times to produce a roughly pure product. The amount of purified T7 core in the circuit was varied over several runs, using a standard of 57 ng miRNA analyte. We found a preferred amount was about 2.2 pg of the T7 core added to the system (data not shown). The purified core produced a higher fluorescence output than was seen with the unpurified core-enriched extract.

Other preferred concentrations and reaction conditions were also assessed (data not shown). A lower cell extract concentration was found to be beneficial in the 3-tiered circuit using the purified T7 core. This system showed higher fluorescence as the concentration of the plasmid expressing the CCG sigma increased, up to at least 9 nM. The effects of the concentrations of each of the plasmids were also examined. Because a cell-free system does not typically replenish the energy and nutrients consumed during transcription and translation, expression in a cell-free system eventually ceases. Such termination can present a problem in complex circuits if too much energy is expended at the beginning of a reaction cycle, leaving too little remaining for later reactions. This problem is particularly pronounced when DNA inputs are used, and when RNA and protein saturation points are reached relatively early due to the depletion of RNases and proteases from the strain BL21 Star DE3. In our preferred CFS circuit, two plasmid DNA inputs are initially transcribed, leaving RNA from both sigma11 and anti-sigma11. Once the analyte is present, it shuts down anti-sigma production at the translational level. Thus RNA saturation levels can be surpassed even when the analyte is present, which results in the system consuming additional energy. Low inputs of these DNA sequences are acceptable, because one plasmid molecule can make many copies of an RNA transcript.

The next plasmid input in one embodiment of the circuit is pJL1-Ps11-CGG, which carries the pseudo-sigma CGG gene. Finding a preferred concentration for the pJL1-Ps11-CGG plasmid presented a more difficult problem: If a low number of analyte molecules are present, then only a small amount of sigma11 will be present to express the CGG sequence; but on the other hand, even a single sigma11 molecule can initiate transcription for multiple CGG sequences.

When one seeks a preferred concentration for this second plasmid, controlling only the amount of activator plasmid (sigma11)—without the repressor plasmid (anti-sigma11) present and without the analyte present—one can potentially reach inaccurate conclusions, due to the difficulty in matching the level of sigma11 freed by the analyte and the conservation of energy at that point. When no RISC or repressor was present, then as the concentrations of the sigma11 plasmid and CGG plasmid varied, the amount of reporter protein mNG (made under the control of CGG) was highest around a 2-4 nM concentration of the CGG plasmid, with relatively small variations resulting from changing the amount of sigma11 plasmid (data not shown).

By contrast, with low levels of the RISC analyte and the repressor plasmid (at a 1:1 ratio with the sigma11 plasmid), when the sigma11 and CGG plasmid concentrations were varied we observed that a higher concentration of CGG plasmid was needed for the same level of expression of the reporter molecule. Without wishing to be bound by this hypothesis, these results suggest that there might be a very low amount of sigma11 freed even at low levels of the RISC, and that it could thus be more beneficial to saturate the promoter binding sites for the freed sigmas, with less concern over the loss of energy due to oversaturation of RNA and protein production at level 2 of the circuit. The optional amplifier molecule relies on the notion that a relatively small amount of freed sigma can induce the expression of a relatively large amount of reporter protein and its strong fluorescence signal. Also, since pseudo-sigma CGG is part of the synthetic SynZip system with a split-T7 core, it is possible that the pairing is not always 100% effective, resulting in a need for higher levels of CGG.

We tested the effect of varying the ratio of initial activator and repressor plasmids in the 3-tier circuit. A ratio of about 1 activator plasmid molecule: 1.15 repressor plasmid molecules was found to result in the highest RFU when the circuit was in the ON state at low levels of miRNA-RISC, measured as a multiple of the RFU seen in the OFF state with no analyte present (data not shown). We also observed that higher levels of the sigma 11 plasmid could reduce measured RFU output, with 0.3 nM of sigma 11 plasmid giving better results than 0.8 nM of sigma 11 plasmid.

### Example 7. RISC Purification from Exosomes

To demonstrate the effectiveness of the novel miRNA sensing tool for potential clinical applications, we purified RISC-miRNA complexes from the exosomes of a breast cancer cell culture having known up-regulators miR-155 and miR-10b. RISC-miRNA complexes are extracellular cargo, and they travel through the bloodstream in an active state in extracellular vesicles (EVs) such as exosomes. We thus concentrated the analyte by first purifying and then lysing the EVs. Methods known in the art, such as ultracentrifugation, filtration, or precipitation, can be used to isolate extracellular vesicles. Polymeric precipitation is becoming more popular, especially with serum/plasma samples and for studying protein and RNA content, because one can obtain high-quality exosomes while maintaining exosome integrity. The exosomes were concentrated from the cell culture media using Total Exosome Isolation (TEI) reagent. To find an efficient route to extract the RISC-miRNA complexes while maintaining their activity, we screened several non-denaturing lysis buffers known in the art, and measured lysing efficiency by nanoparticle tracking analysis (NTA); by Western blot targeting the hAGO2 protein; and by assaying RISC activity by adding the sample to our circuit. Transmission Electron Microscopy imaging (TEM) and Dynamic Light Scattering (DLS) were performed to verify that exosome purification from the breast cancer cell culture had been successful. Following lysis, the samples are added to the circuit reaction mixtures to detect the different types of miRNA that might have been present in the samples, and the fluorescence was recorded.

To enhance the signal further, the duration of the cell-free protein synthesis reactions may optionally be extended using a cell-free dialysis system, using a dialysis system known in the art. One example of such a system, sometimes known as a cell-free continuous exchange (CFCE) system, includes a housing with two chambers separated by a membrane. The CFCE system includes a cell-free reaction chamber on one side with appropriate components for a batch reaction, and a feed chamber on the other side also containing most of the appropriate components, but lacking the input DNA and the cell extract. The feed chamber may, for example, be three to ten times greater in volume than the cell-free reaction chamber. Such housing devices can be made, for example, using 3D printing techniques known in the art, with the option of allowing either horizontal dialysis transfer or vertical dialysis transfer. A CFCE system can allow longer run times than a simple batch system, allowing more RNA and more protein to be made from the plasmids (which themselves are relatively stable). These longer run times can extend the reaction from −6 hours to −72 hours, and can increase protein production by ˜600%, by continually feeding new nutrients from the feed chamber into the reaction chamber. To maintain the flow of new nutrients, the feed chamber can be emptied and re-supplied as needed. Resupply can occur, for example, every 6-12 hours. Furthermore, inhibitory byproducts, such as inorganic phosphate, should tend to migrate from the reaction chamber.

### Example 8. Plasmid and Strain Information

All plasmids were made by the Gibson Assembly protocol, following the manufacturer's instructions. The plasmids used for comparing sigma factors were made using the pJL1 vector backbone, and amplifying the sigma genes obtained from Addgene Plasmids; or by ordering the fragment as a gBlock from Integrated Gene Technologies (IDT). Plasmids with genes transcribed by T7 RNA polymerase contained a T7 promoter and terminator. Plasmids with genes transcribed by a sigma factor and endogenous RNA polymerase contained the cognate promoter for the plasmid being tested and the t500 terminator.

One embodiment of the simple gene circuit comprised four plasmids: (1) pJL1-T7-AntiSigmaECF11, expressing anti-sigma ECF11 (687 nt, 25 kDa), with coding sequence from V. A. Rhodius et al., Design of orthogonal genetic switches based on a crosstalk map of σs, anti-σs, and promoters. Molecular systems biology 9, 702-702 (2013). (2) pJL1-T7-SigmaECF11, expressing ECF11_987 Sigma gene (585 nt, 22 kDa), with coding sequence from V. A. Rhodius et al. (2013). (3) The reporter plasmid pJL1-Ps11-mNG, expressing the mNG sequence coding sequence from L. Hostettler et al., The Bright Fluorescent Protein mNeonGreen Facilitates Protein Expression Analysis In Vivo. G3 (Bethesda) 7, 607-615 (2017), and codons optimized using the Integrated DNA Technologies (IDT) tool. The sequences 1-3 noted here were purchased from IDT as DNA fragments (gBlocks).

The plasmids used in the amplifier circuit comprised: (4) pJL1-T7-CGGsig, expressing the CGG sigma fragment (996 nt, 37 kDa), with coding sequence obtained from V. A. Rhodius et al. (2013). (5) pJL1-Ps11-CGGsig, expressing the same CGG sigma fragment, but with the sigma ECF11 promoter. (6) pJL1-PCGG-mNG, expressing the mNG coding sequence under the CGG promoter, with the promoter sequence obtained from T. H. Segall-Shapiro et al., A ‘resource allocator’ for transcription based on a highly fragmented T7 RNA polymerase. Molecular Systems Biology 10, 742 (2014). (7) The T7-split core fragment plasmid was obtained from Addgene (pTHSSd_38, #59961); however, it was found to be incompatible with our system, so the core fragment was instead inserted into a Novagen pETBlue-1 expression vector. (8) The pJL1 Anti-SigmaECF11 plasmid, containing a complementary miRNA-RISC binding site a few nucleotides after a stop codon. This position was screened to avoid any “inadvertent” stop codons or probable frameshifts.

The bacteria Escherichia coli BL21 Star™ (DE3), genotype F-ompT hsdSB (rB−, mB−) galdcmrne131 (DE3), was used to prepare the cell extract for the cell-free protein synthesis reactions. This strain contains a coding sequence for T7 RNA polymerase controlled by the lacUV5 promoter, which can be induced by IPTG. It is also deficient in RNase E and in the proteases Ion and Omp-T, in order to reduce degradation of mRNA and protein.

### Example 9. Cell Extract Preparation

The cell extract used in prototype examples was made by combining the protocols of: (1) Y.-C. Kwon et al., High-throughput preparation methods of crude extract for robust cell-free protein synthesis. Scientific Reports 5, 8663 (2015); (2) J. Kim et al., A Crude Extract Preparation and Optimization from a Genomically Engineered Escherichia coli for the Cell-Free Protein Synthesis System: Practical Laboratory Guideline. Methods and protocols 2 (2019); and (3) A. D. Silverman et al., Deconstructing Cell-Free Extract Preparation for in Vitro Activation of Transcriptional Genetic Circuitry. ACS Synthetic Biology 8, 403-414 (2019).

The overall procedures are broadly similar across these three protocols, with some individual differences. Briefly, cells were grown overnight in 2xYTPG media (from J. Kim et al. (2019)); and the optical density at 600 nm was monitored until the culture reached mid-exponential phase growth, OD 1.8. The cells were then harvested and washed three times with S30 buffer (from A. D. Silverman et al. (2019)) with magnesium and potassium glutamate at pH 7.7. After washing, the pellets were frozen and stored at −80° C. Later, the cells were resuspended in the Silverman et al. S30 resuspension buffer, pH 8.2, and lysed by sonication with a Q125 Sonicator with a ⅛″ (3 mm) diameter probe (Qsonica, Newtown, CT, USA). The following energy equation from Y.-C. Kwon et al. (2015) was used to estimate the output in joules per unit cell suspension volume: [Energy]=[Volume (μL)]−33.6]×1.8⁻¹. The lysates were then centrifuged, and the supernatant was separated and used for the cell-free extract. Post-lysis steps were continued following the procedures in A. D. Silverman et al. (2019): Run-off reaction was performed by incubating the clarified lysate at 37° C. with shaking at 200 rpm for 80 min., followed by centrifugation to clarify the lysate. The supernatant was dialyzed using a 10K MWCO dialysis cassette in S30 buffer at pH 8.2 for 2.5 hours, with no buffer changes. Then the dialyzed lysate was centrifuged, and the supernatant was collected and divided into aliquots. The aliquots were flash-frozen in liquid nitrogen and stored at −80° C. Other cell-free expression systems known in the art may also be used in practicing the invention.

### Example 10. Split-T7 Core Expression and Purification

The T7 core split piece sequence from Addgene plasmid pTHSSd_38, #59961, which was published in connection with T. H. Segall-Shapiro et al., A ‘resource allocator’ for transcription based on a highly fragmented T7 RNA polymerase. Molecular Systems Biology, 2014. 10(7): p. 742, was inserted into a pETBlue1 vector with an N-terminus histidine tag after the start codon, using the Gibson Assembly protocol. The resulting plasmid was transformed into a competent strain of E. coli BL21 Star™ (DE3), genotype F-ompT hsdSB (rB−, mB−) galdcmrne131 (DE3). This strain contains the coding sequence for T7 RNA polymerase under control of the lacUV5 promoter, which can be induced by IPTG. The transformant was plated on a Carbenicillin 50 μg/mL antibiotic resistance plate. Crude cell extract containing the overexpressed protein could then be obtained, or whole cell expression could be carried out for protein purification. For crude extract preparation, the cells were cultured and processed as otherwise described above, except that smaller, 125 mL cultures were used, because we had found that the extracts from 125 mL cultures showed greater activity than those from larger cultures (data not shown). For comparisons, samples were taken before run-off and dialysis, after run-off and before dialysis, and after both run-off and dialysis.

For protein purification, a colony was selected and grown overnight in 5 mL LB media culture, 37° C. with shaking at 250 rpm. The next day, the culture was inoculated into a larger, 500 mL LB media culture at a 1:500 dilution. The culture was then allowed to grow at 37° C. with shaking at 300 rpm in a 2.5 mL Tunair shake flask. Once the culture had reached OD 0.6, 1 mM final concentration of IPTG was added to the culture to induce T7RNAP expression and subsequent expression of the T7 core split protein from the pETB1 plasmid. The temperature was then lowered to 20° C., and the flask was shaken at 250 rpm overnight. The next day the culture was harvested and stored at −80° C. until used. The cell pellet was then resuspended at a 1:1 mass ratio of lysis buffer to pellet. The suspension was aliquoted into 1 mL tubes, and sonicated with 537 Joules per mL lysate. The lysate was then clarified by centrifugation at 12,000 RCF at 4° C. for 15 minutes. The supernatant was saved and subjected to His-tag purification.

His-tag purification was performed using Qiagen Ni-NTA resin, per the manufacturer's protocol. The resin was washed 5 times with 40 mM imidazole in the wash buffer, and then eluted with 250 mM imidazole in the elution buffer. The eluate was concentrated by centrifuge with 10 kDa MWCO Amicon centrifugal filters. The buffer was replaced with a storage buffer that does not interfere with the cell-free reaction. The storage buffer comprised 20 mM sodium phosphate, pH 7.7, 1 mM EDTA, 1 mM DTT, 5% glycerol, and 100 mM NaCl. The purified protein was then aliquoted into 25 μL portions, and flash-frozen in liquid nitrogen. This storage method preserved the activity of the purified protein. Once a sample was thawed, it was not re-frozen and saved for additional use, because its activity would be diminished or lost by repeated cycles of freezing and thawing.

### Example 11. RISC Loading

Flag-tagged human Argonaute2 (Ago2) protein was synthesized using a baculovirus expression system in Sf9 cells. The cells were lysed with a Kontes Dounce tissue grinder and lysis buffer. Then Ago2 was purified using an anti-flag M2 affinity gel, with a competing FLAG peptide, both purchased as a kit from Sigma Aldrich. The eluted protein was stored at 4° C. and used within 2 weeks. Single-stranded guide microRNA-155 (5p) (ss-miRNA-155) with the 5′ end phosphorylated was purchased from IDT. The loading reaction comprised RISC cleavage buffer (1 unit/μL RNasin, 20 mM Tris, 50 mM KCl, 5% glycerol, 1.5 mM MgCl₂), 200 nM Ago2, 1750 nM ss-miR-155, and nuclease-free water to make the final volume 2.15 μL. The mixture was incubated at 37° C. for 30 minutes to allow RISC formation.

### Example 12. Cell-Free Protein Synthesis

Cell-Free Protein Synthesis (CFPS) reaction mixtures were prepared following the methods of J. Kim et al. (2019), including a salt mixture, NTPs, folinic acid, tRNA, HEPES, NAD, CoA, oxalic acid, putrescine, spermidine, PEP, 20 essential amino acids, T7 RNA polymerase, S30 cell-extract as described in Example 9, and plasmids as described in Example 8 (in various combinations). The final volume was 15 μL. The reaction tubes were then incubated at 37° C. for 20 hours. For circuit reactions, the following reagents were added: 2.15 μL of RISC formation product, 0.2 nM of pJL1-T7-SigmaECF11, 0.025 nM pJL1-T7-AntiSigmaECF11, and 13 nM of pJL1-Ps11-mNG. As a positive control for the circuit, 2.15 μL of RISC formation product was added along with 0.2 nM of pJL1-T7-SigmaECF11 and 300 ng of pJL1-s11-mNG. Negative control for the circuit used all the simple circuit plasmids, but without any RISC formation product. Negative control for the reaction had no DNA added. Negative control for the reporter protein was 13 nM of pJL1-Ps11-mNG and 2.15 μL of RISC formation product. For the amplifier circuit, the same reagents were used, except that 0.5 nM of pJL1-Ps11-CGGsig, 13 nM of pJL1-PCGG-mNG (and no pJL1-s11-mNG), and 2.5 pg of purified T7 Split core piece (or 3 μL of enriched T7 Split core crude cell extract for initial tests) were added.

### Example 13. Protein Analysis

After 20 hours reaction time, fluorescence was measured with a Synergy™ HTX multi-mode microplate reader (BioTek, Winooski, VT, USA) with excitation and emission wavelengths of 485 nm and 528 nm, respectively. The remaining reaction mixture was used to analyze total protein, soluble protein, and insoluble protein, using SDS-PAGE (NuPAGE® 4-12% bis-tris gel (Thermo Fisher Scientific) to separate proteins by molecular weight. A standard curve was generated for RFU as a function of protein concentration over a series of consecutive dilutions. Protein concentration was determined by overexpressing mNG in BL21 DE3 Star culture, purifying with the Strep tag attached to the protein, and determining the concentration using a BSA standard.

### Example 14. RT-qPCR and Efficiency. LOD Comparisons

The miRNA loading reactions were carried out as described in Example 10, performed separately for each miRNA type. The products of the loading reaction were immediately added to the start of the sensing reaction cascade and incubated at 37° C. For RT-qPCR reactions, the miRNA was first purified from the AGO protein after the loading reaction, in the same manner as a liquid biopsy sample would ordinarily be processed. A Qiagen miRNeasy Kit (cat. no. 217084) was used to purify each miRNA type. A 1 μL sample of a control miRNA (˜108 copies), UniSp6 RNA, was spiked into each tube at the beginning of the purification to serve as a qPCR reference gene. After purification, the miRNAs were separately reverse-transcribed into cDNA using the miRCURY LNA RT Kit (cat. no. 339340). Afterwards, the reaction was diluted 1:30 for further qPCR reactions. miRCURY LNA miRNA PCR Assays (cat. no. 339306) and 2× miRCURY SYBR® Green Master Mix (cat. no. 339345) were purchased from Qiagen. For specificity tests, miR-155 primer was used for the miRNA cDNA template. UniSp6 RNA primers were also used to normalize the results. RFU and Cq values were determined.

### Example 15. Calculation of LOD and LOQ

The limit of detection (compared to a blank sample) was found to be 81 pM, and the limit of quantification was found to be 271 pM. However, a comparison to a blank reaction (i.e., one with no miRNA) may not be a good representation for a “real world” sample that would more likely contain a variety of miRNAs, perhaps including some homologous sequences that are similar to but not identical with the target sequence. To provide a better measure of LOD and LOQ for “real world” assays, the system's response to an miRNA with a single base mismatch was substituted as the “blank” reaction. In the presence of the single-base-mismatch “contaminant,” the LOD was found to be 161 pM, and the LOQ was found to be 537 pM.

The LOD was again determined as the point where the signal/noise ratio was 3, and the LOQ as the point where the ratio was 10. In the following equations, μ_{b}=mean signal of the blank, σ_{b}=standard deviation of the blank, LOD=limit of detection of the reporter, LOQ=limit of quantification of the reporter:

$$LOD = {μ}_{b} + 3 * {σ}_{b}$$

$$LOQ = {μ}_{b} + 10 * {σ}_{b}$$

### Example 16. Freeze-Drying the CFE Components and DNA

Cell-free components may be freeze-dried into pellets for on-demand use and room temperature storage, following the method of J. K. Jung et al., Cell-free biosensors for rapid detection of water contaminants. Nature Biotechnology, 2020. 38(12): p. 1451-1459. Briefly, the reaction mixtures are assembled with all components, for example as described above in Example 11. Cryopreservatives are added to the mixture in PCR tubes, for example 50 mM sucrose and 250 mM D-mannitol. The PCR tubes have three holes punched in their caps to aid in lyophilization. Then the tubes are inserted in a pre-chilled block tube-holder, and placed in a −80° C. freezer for 10 min. After the initial freeze, the tubes are wrapped in Kimwipes and aluminum foil, submerged in liquid nitrogen, and transferred to a freeze dryer system for lyophilization overnight at −85° C. and 0.04 mbar pressure. We have successfully prepared prototype embodiments as freeze-dried systems, on small Whatman paper discs. The reactions from the prototypes on paper resulted in high fluorescence levels. In many applications, freeze-dried pellets would be preferred over paper.

### Example 17. Statistical Analyses

Statistical analyses were conducted using Graphpad Prism 8.4.3 (GraphPad Software) at a 5% significance level. For parametric analysis of data from the quantification of the synthesized protein, we used two-way ANOVA followed by Dunnett's test.

### Miscellaneous

The complete disclosures of all references cited herein are hereby incorporated by reference in their entirety. Also incorporated by reference is the complete disclosure of the following work by the present inventors: C. Copeland and Y. Kwon, “Using the Cell-Free Expression System for Highly Accurate Microrna Detection,” presentation at the AIChE Annual Meeting, Nov. 18, 2022); and the associated Abstract (Abstract available online from Jul. 15, 2022). Also incorporated by reference is the complete disclosure of the following work by the present inventors: C. Copeland and Y. Kwon, “Retaining Accuracy in miRNA Detection Using the Cell-Free Gene Expression System,” presentation at the Louisiana State University AgCenter Symposium 3.0 (Mar. 23, 2023) Also incorporated by reference is the complete disclosure of the priority application, U.S. provisional patent application Ser. No. 63/425,509, filed 15 Nov. 2022. In the event of an otherwise irreconcilable conflict, however, the present specification shall control over material incorporated by reference.

What is claimed:

1. A method for detecting the presence or absence of a specified target micro RNA sequence in a sample; said method comprising the steps of:
(a) supplying RNA-induced silencing complexes (RISCs) from the sample, under conditions that preserve the binding of any micro RNA to the RISCs;
(b) mixing the RISCs, along with any micro RNA that is bound to the RISCs, with a cell-free protein expression system; and
(c) detecting a signal from the cell-free protein expression system that indicates the presence of the target micro RNA sequence in the sample, or observing the absence of such a signal as indicating the absence of the target micro RNA sequence from the sample.

2. The method of claim 1, wherein:
(a) the cell-free protein expression system comprises a DNA sequence that encodes an mRNA sequence complementary to the target micro RNA sequence, wherein said DNA sequence is operatively linked to a sequence encoding an anti-sigma protein; wherein expression of the anti-sigma protein is disrupted by binding of the target micro RNA and RISC to the complementary mRNA sequence when the target micro RNA sequence is present in the extracted RISCs; and wherein the anti-sigma sequence is expressed without disruption when the target micro RNA sequence is absent from the extracted RISCs; and
(b) a reporter molecule is expressed at detectable levels when the level of anti-sigma protein in the cell-free protein expression system is thereby down-regulated by the presence of the target micro RNA sequence; and the reporter molecule is not expressed at detectable levels when transcription of the anti-sigma protein remains undisturbed in the absence of the target micro RNA sequence.

3. The method of claim 2, wherein said method is sufficiently sensitive to distinguish the specified target micro RNA from an off-target micro RNA that differs at only one nucleotide.

4. The method of claim 2, wherein the cell-free protein expression system comprises a nucleic acid encoding a sigma protein that is non-native to the cell-free protein expression system; wherein the anti-sigma protein is complementary to the non-native sigma protein; and wherein expression of the reporter molecule is dependent on the presence of non-native sigma protein that is unbound to complementary anti-sigma protein.

5. The method of claim 4, additionally comprising the step of amplifying the expression of the reporter molecule in response to the presence of unbound, non-native sigma protein.

6. The method of claim 5, wherein the cell-free protein expression system comprises the core component of T7 RNA polymerase; and wherein said amplifying step comprises expressing an mRNA sequence that encodes a pseudo-sigma component of T7 RNA polymerase under the control of a promoter responsive to the presence of unbound, non-native sigma protein; and wherein the core component and the pseudo-sigma component bind to one another when both are present in the expression system, thereby producing a functional T7 RNA polymerase; and wherein the functional T7 RNA polymerase in turn binds to a cognate promoter controlling expression of the reporter molecule, thereby amplifying expression of the reporter molecule.

7. The method of claim 2, wherein at least some of the reagents used in performing said method are first lyophilized and then rehydrated.

8. A method for detecting the presence or absence of a specified RNA sequence in a sample; said method comprising the steps of:
(a) loading RNA from the sample into Argonaute protein molecules to generate synthetic RNA-induced silencing complexes (RISCs);
(b) adding the synthetic RISCs, along with any RNA bound to the RISCs, to a cell-free protein expression system; and
(c) detecting a signal from the cell-free protein expression system that indicates the presence of the target RNA sequence in the sample, or observing the absence of such a signal as indicating the absence of the target RNA sequence from the sample.

9. The method of claim 8, wherein:
(a) the cell-free protein expression system comprises a DNA sequence that encodes an mRNA sequence complementary to the target RNA sequence, wherein said DNA sequence is operatively linked to a sequence encoding an anti-sigma protein; wherein expression of the anti-sigma protein is disrupted by binding of the target micro RNA and RISC to the complementary mRNA sequence when the target RNA sequence is present in the RISCs; and wherein the anti-sigma sequence is expressed without disruption when the target RNA sequence is absent from the RISCs; and
(b) a reporter molecule is expressed at detectable levels when the level of anti-sigma protein in the cell-free protein expression system is thereby down-regulated by the presence of the target RNA sequence; and the reporter molecule is not expressed at detectable levels when transcription of the anti-sigma protein remains undisturbed in the absence of the target RNA sequence.

10. A kit for detecting a target micro RNA sequence, said kit comprising:
(a) a lyophilized cell-free protein synthesis system;
(b) a lyophilized DNA sequence that encodes an mRNA complementary to the target micro RNA sequence, and that encodes an anti-sigma protein, wherein said mRNA complementary to the target micro RNA sequence is upstream of the sequence encoding the anti-sigma protein;
(c) a lyophilized DNA sequence encoding a non-native sigma protein;
(d) a lyophilized DNA sequence encoding a pseudo sigma protein;
(e) a lyophilized DNA sequence encoding a core T7 RNA polymerase; wherein neither the pseudo sigma protein alone nor the core T7 RNA polymerase alone is active as a polymerase; and wherein, in the presence of both components, the pseudo sigma protein and the core T7 RNA polymerase bind to one another to produce a functional T7 RNA polymerase; and
(f) a lyophilized DNA sequence encoding a reporter protein.

11. The kit of claim 10, additionally comprising lyophilized Argonaute protein and associated loading reagents.
